(19) United States
(12) Patent Application Publication
(10) Pub. No.: US 2008/0206293 A1
(43) Pub. Date: Aug. 28, 2008

(54) Absorbent substrate with a non-leaching antimicrobial activity and a controlled-release bioactive agent.

(75) Inventors: William Toreki, Gainesville, FL (US); Bernd Liesenfeld, Gaines, FL (US); David Moore, Gainesville, FL (US); Gregory Schultz, Gainesville, FL (US); Gerald Olderman, Bedford, MA (US); Gregory Staab, Ventura, CA (US)

Correspondence Address: ELMAN TECHNOLOGY LAW, P.C., P. O. BOX 209, SWARTHMORE, PA, 19081, US

(73) Assignees: QUICK-MED TECHNOLOGIES, INC., Gainesville, FL (US); UNIVERSITY OF FLORIDA RESEARCH FOUNDATION, INC., Gainesville, FL (US)

(21) Appl. No.: 12/036,107

(22) Filed: Feb. 22, 2008

Related U.S. Application Data
(63) Continuation of application No. PCT/US2006/032953, filed on Aug. 22, 2006.
Application No. PCT/US2006/032953 is a division of application No. 10/786,959, filed on Feb. 25, 2004.
(60) Provisional application No. 60/710,131, filed on Aug. 22, 2005.

(51) Int. Cl. A61K 9/00; A61F 13/00; A61K 31/65; A61K 38/18; A01N 25/08; A01P 1/00; A61P 31/04; A61K 38/02; A01N 25/00; A61K 31/165; A61K 31/14

(52) U.S. Cl. 424/404; 424/445; 514/154; 514/642; 424/446; 514/152; 514/12; 424/405; 424/411; 424/412; 424/400; 514/2

(57) ABSTRACT

This invention relates to antimicrobial wound dressings having a non-leaching antimicrobial activity, releasable antimicrobial and antiprotease agents, and a controlled-release bioactive agent such as doxycycline. The Wound dressing material is absorbent and acts as a substrate for antimicrobial and antiprotease agents as well as bioactive agents. More generally, this invention relates to methods and compositions for materials having a non-leaching coating that has antimicrobial properties. The coating is applied to substrates such as gauze-type wound dressings, powders and other substrates. Covalent, non-leaching, non-hydrolyzable bonds are formed between the substrate and the polymer molecules that form the coating. A high concentration of anti-microbial groups on multi-length polymeric molecules and relatively long average chain lengths, contribute to an absorbent or superabsorbent surface with a high level antimicrobial efficacy. Utilization of non-leaching coatings having a plurality of anionic or cationic sites is used according to this invention to bind a plurality of oppositely charged biologically or chemically active compounds, and to release the bound oppositely charged biologically or chemically active compounds from said substrate over a period of time to achieve desired objectives as diverse as improved wound healing to reduction in body odor.

### CROSS REFERENCE TO RELATED APPLICATIONS

This application is a divisional of our co-pending application Ser. No. 10/786,959 filed Feb. 25, 2004 and claims benefit of priority to that application. The present application is also a continuation of our co-pending International Application, Ser. No. PCT/US2006/032953, filed Aug. 22, 2006, which is a non-provisional of U.S. Provisional Application No. 60/710,131, filed Aug. 22, 2005 and claims benefit of priority to both prior applications. The disclosures of our U.S. application Ser. No. 10/786,959, our International Application No. PCT/US2006/032953, and our Provisional Application No. 60/710,131 are hereby incorporated by reference.

### FIELD OF THE INVENTION

This invention relates to wound dressings, more particularly to wound dressings having a non-leaching antimicrobial activity and providing for the controlled-release of bioactive substances. This invention also relates to methods and compositions for materials having a non-leaching coating which provide controlled release of biologically active substances from select substrates.

### BACKGROUND ART

Absorbents such as carboxymethyl cellulose (CMC) and alginates are commonly used in wound dressings to absorb exudate fluids and cellular debris. This absorbed wound exudate fluid is an ideal medium for microbial growth, and this microbial growth can be detrimental to wound healing in at least two ways. First, toxins produced by microorganisms growing in the wound dressing may diffuse into the wound and impair the health of eukaryotic cells. Second, microorganisms growing on the dressing may be shed into the wound and result in higher microorganism colonization, ultimately leading to infection of the wound. Even sub-clinical infections, defined as less than one million colony forming units per ml, have been shown to significantly impair or impede wound healing.

Some commercially available moist wound dressings endeavor to prevent the growth of microorganisms by incorporating a diffusing biocidal agent. One commonly used diffusing biocidal agent is silver. One disadvantage to this approach is that certain bacteria have been able to develop resistance to silver. (Silver S., “Bacterial silver resistance: molecular biology and uses and misuses of silver compounds.” FEMS Microbiology Reviews, 2003; 27:341-353). Another disadvantage to this approach is that diffusing silver may be able to enter the wound and may potentially stain the skin. An additional disadvantage of silver is the high cost of the raw material.

Several approaches to wound management rely on the release of antimicrobial agents such as silver ions. Burton et al. (US_{—}2005/0124724_A1) disclose a polymer composition comprising: a hydrophilic polymer; and a bioactive agent selected from the group consisting of a metal oxide of silver, copper, zinc, and combinations thereof; wherein the bioactive agent is dispersed within the hydrophilic polymer; and wherein substantially all of the bioactive agent has a particle size less than one micron. Cooper et al. (US_{—}2005/0008840_A1) disclose an antimicrobial material and a method of inhibiting infection during the treatment of a wound by applying an antibacterial material to an affected skin area on a patient, said antibacterial material including: a fibrous substrate impregnated with a carrier and a controlled release biocide, e.g. triclosan, dispersed through the carrier. The substrate is desirably a substantially cellulose fiber blend, and the biocide is desirably non-allergenic to humans, and inhibits the growth of E. coli, Legionella or Staphylococcus. Munro et al. (WO_{—}04/028255_A1) disclose an antimicrobial composition comprising (a) an antimicrobially effective amount of a dissolved antimicrobial metal ion and (b) a dissolved halide ion. Bowler et al. (US 2004/0001880_A1) disclose the use of an effective amount of silver in the manufacture of a wound dressing comprising an anionic, amphoteric or hydrophilic polymer, which dressing, when applied to a wound site, gives a controlled release of ionic silver into the wound fluid for the prevention of staining of the underlying tissue. Gibbins et al. (U.S. Pat. No. 6,897,349) disclose a hydrophilic antimicrobial fiber comprising a hydrophilic polymer, such as carboxymethyl cellulose, of a wound dressing for external application to a wound, wherein the hydrophilic polymer of the wound dressing contains a silver salt compound.

There are also numerous examples which provide for wound dressings with releasable bioactive agents (other than antimicrobials). Yamazaki et al. (U.S. Pat. No. 5,098,417) disclose a wound dressing for systemic administration of a physiologically- or biologically-active agent by controlled release of the agent into such wound, the wound dressing comprising a substrate in the form of a fabric or cloth, at least a portion of which is cellulosic, which has been chemically modified to convert hydroxyl groups in said cellulosic portion to ionic-adsorbing sites; an ionic form of a physiologically- or biologically-active agent adsorbed in said substrate, namely an antibacterial agent, an antifungal agent, an analgesic agent, a tissue healant agent, a local anesthetic agent, an antibleeding agent, an enzyme or a vasoconstrictor. The patent teaches that ionic bonds hold the agent temporarily to the substrate for controlled release therefrom in proportion to the amount of exudate in contact with the substrate. The ionic bonds are formed by adsorbing the agent on the substrate at room temperature, the ionic bonds disassociating upon contact with body exudate from wounds to which the wound dressing is applied by ion exchange with ions in the body exudate, thereby to release the physiologically- or biologically-active agent in an amount in proportion to the amount of the exudate in contact with the substrate.

Silcock et al. (WO 04/080500 A1) disclose a wound dressing material comprising a low-moisture hydrogel matrix having oxidized cellulose distributed therein, and wherein the hydrogel matrix comprises a hydrogel selected from the group consisting of modified celluloses, modified starches, alginates, plant gums, gelatins, glycosaminoglycans, polyacrylates, polyurethanes, and mixtures thereof, and wherein the hydrogel is selected from the group consisting carboxymethyl cellulose salts, alginate salts, gelatins, hyaluronic acid and its salts, xanthan gum, guar gum, and mixtures thereof, and wherein said wound dressing material further comprises one or more therapeutic agents. Bootman et al. (EP 1120112 A2) disclose a wound dressing and drug-delivery device for controlled-release of a bioactive agent comprising a cross-linked biopolymer and a bioactive agent reversibly bound thereto, and wherein said biopolymer comprises collagen or gelatin, and wherein said bioactive agent comprises silver ions, and wherein said bioactive agent comprises a peptide and/or protein, growth factor, or immune-modulating factor. Soerens et al. (WO 04/011046 A1) disclose an absorbent binder composition comprising a water-soluble ionic polymer capable of sufficient non-radiative crosslinking within about 10 minutes at a temperature of about 120° C. or less, to reach an absorbent capacity of at least one gram per gram, and wherein the ionic polymer comprises a carboxyl group-containing monomer, and wherein the ionic polymer comprises a quaternary ammonium group-containing monomer, and wherein the binder is applied as a coating to a substrate, and wherein the substrate comprises a person's skin, an article of clothing, a wound dressing, and wherein the absorbent binder coating further comprises at least one of a fragrance additive, odor-absorbing particles, an antimicrobial additive, a wound healing agent, or a fungicide. Berthold et al. (U.S. Pat. No. 6,399,091) disclose a wound dressing having a layered structure for the controlled release of active substance to wounds comprising two polymer-containing layers each comprising a hydrocolloid-containing swellable hydrogel as an absorbent; two woven layers; and at least one active substance in at least one of the layers, the polymer-containing layers and the woven layers being superposed in alternating sequence, and wherein the polymer is a cellulose, such as carboxymethyl cellulose, and wherein the active substance is a biologically active peptide, protein, or growth factor, and wherein the woven layer is based on polyester, polyurethane or cellulose.

Other approaches combining a non-leaching antimicrobial activity with a releasable bioactive agent include the following. Toreki et al. (US 2005/0033251 A1) disclose a material comprising a substrate and an enhanced surface area, the enhanced surface area comprising a multitude of non-hydrolyzable, non-leachable polymer chains covalently bonded by non-siloxane bonds to said substrate; wherein said non-hydrolyzable, non-leachable polymer chains comprise a multitude of antimicrobial groups attached to said non-hydrolyzable, non-leachable polymer chains by covalent bonds; and wherein a sufficient number of said non-hydrolyzable, non-leachable polymer chains are covalently bonded to sites of said substrate to render the material antimicrobial when exposed to aqueous fluids, menses, bodily fluids, skin, cosmetic compositions, or wound exudates, wherein said material has associated therewith a plurality of anionically charged biologically or chemically active compounds, and wherein said antimicrobial groups comprise at least one quaternary ammonium structure. The disclosed substrate is comprised, in whole or in part, of cellulose, or other naturally-derived polymers or comprises all or part of a wound dressing.

Proteases are enzymes that have catalytic activity (that is typically highly specific) against certain proteins. Proteases help mediate the degradation of protein based structures, such as tissue structures the body needs to remodel, as occurs in wound healing, the blood clotting cascade, and in apoptosis pathways. In the case of chronic wounds the protease concentration at the wound site is out of balance, leading to retardation or complete arrest of wound healing because the rate of tissue degradation (protease mediated) overwhelms the rate of tissue formation. Protease inhibitors have been applied with significant success to aid the healing and closure of chronic wounds. Protease inhibitors bind proteases and therefore prevent their action. A simpler approach to protease control that is used due to simplicity is to provide a sacrificial substrate such as the mixture of collagen and oxidized regenerated cellulose of Johnson and Johnson's Promogran™.

There exists a need for an absorbent material that can resist colonization by microorganisms and can additionally provide for the controlled-release of a bioactive substance.

### DISCLOSURE OF THE INVENTION

The current invention provides a device for the treatment of wounds which is comprised of an absorbent wound dressing material having inherent non-leachable antimicrobial and inherent non-leachable anti-protease activity or agent incorporated therein. In addition, the device also contains releasable antimicrobial and releasable anti-protease agents that are ionically-stabilized within the wound dressing, and may be released from the device in a controlled manner. In addition, the device also contains releasable bioactive agents which aid in wound healing, such as growth factors, vitamins, and/or nutrients.

“Microbe” or “microorganism” refers to any organism or combination of organisms such as bacteria, viruses, protozoa, yeasts, fungi, molds, or spores formed by any of these.

“Antimicrobial” refers to the microbicidal or microbistatic properties of a compound, composition, article, or material that enables it to kill, destroy, inactivate, or neutralize a microorgamism; or to prevent or reduce the growth, ability to survive, or propagation of a microorganism.

By “polymeric quaternary ammonium” is meant a polymer wherein multiple quaternary ammonium moieties are covalently bonded to the polymer molecule, or attached to the molecular structure by covalent chemical bonds and form part of the polymer molecular structure, and that said multiple quaternary ammonium moieties are located in the main-chain of the polymer or in side-groups of the polymer. The terms “main-chain” and “side-groups” are commonly used to describe polymer molecular structure and are familiar to one skilled in the art. Sufficient negative counter-ion such as chloride is typically present to balance the positive charge of the quarternary ammonium moieties.

“Polyquaternium” refers to a polymeric quaternary ammonium substance as the term is used in the International Nomenclature of Cosmetic Ingredients (INCI).

By “hydrogel” is meant a gel structure comprised of a crosslinked network structure of a polymer, such as a polymeric quaternary ammonium molecule and water, or a gel formed by a polyelectrolyte complex or network upon absorption of water.

“Incorporated” means bonded to a substrate in a non-leachable manner.

“Inherent non-leachable” means that the agents responsible for the particular effect (such as antimicrobial, anti-protease, or bioactive agents) are immobilized within the wound dressing, and are not eluted or leached when exposed to aqueous fluids, menses, bodily fluids, skin, cosmetic compositions, or wound exudates.

“Releasable” means that the agents responsible for the particular effect (such as antimicrobial, anti-protease, or bioactive agents) may be eluted, leached, or migrate from the wound dressing when exposed to aqueous fluids, menses, bodily fluids, skin, cosmetic compositions, or wound exudates.

“Ionically-stabilized” means that the agents (such as antimicrobial, anti-protease or bioactive agents) show a propensity to remain within the wound dressing as a result of ionic interactions between a component of the wound dressing and the agent, resulting in the retention of the agent within the dressing to a greater extent than would otherwise be expected. Ionic stabilization manifests as decreased solubility or a decreased diffusion rate for the stabilized agent.

“Controlled manner” means that the release of the ionically-stabilized agents (such as antimicrobial, anti-protease or bioactive agents) are released from the wound dressing in a manner that may be controlled or adjusted via appropriate modifications to the composition of the dressing or use conditions, or that the release rate of the ionically-stabilized agents is reduced compared to non stabilized agents.

A “cellulose-based material” means a natural material made in whole or in part of cellulose or a synthetic material derived from cellulose or having chemical and physical properties similar to cellulose.

“Substantially carboxymethylated cellulosic substrate” means a cellulose-based material that has been derivitized to contain carboxymethyl groups at a degree of substitution greater than 0.25. “Degree of substitution” refers to the number of carboxymethyl groups per anhydroglucose unit”.

By “non-hydrolyzable” is meant a bond that does not hydrolyze under standard conditions to which a bond is expected to be exposed under normal usage of the material or surface having such bond. For instance, in a wound dressing according to the present invention that has “non-hydrolyzable” bonds, such “non-hydrolyzable” bonds do not hydrolyze (e.g., undergo a hydrolysis-type reaction that results in the fission of such bond) under: normal storage conditions of such dressing; exposure to would exudates and/or body fluids when in use (e.g., under exposure to an expected range of pH, osmolality, exposure to microbes and their enzymes, and so forth, and added antiseptic salves, creams, ointments, etc.). The ranges of such standard conditions are known to those of ordinary skill in the art, and/or can be determined by routine testing.

By “non-leaching” is meant that sections of the polymer of the present invention do not appreciably separate from the material and enter a wound or otherwise become non-integral with the material under standard uses. By “not appreciably separate” is meant that no more than an insubstantial amount of material separates, for example less than one percent, preferably less than 0.1 percent, more preferably less than 0.01 percent, and even more preferably less than 0.001 percent of the total quantity of polymer. Alternately, depending on the application, “not appreciably separate” may mean that no adverse effect on wound healing or the health of an adjacent tissue of interest is measurable.

In regard to the above, it is noted that “non-leachable” refers to the bond between the polymer chain and the substrate. In certain embodiments of the present invention, a bond between the polymer backbone and one or more type of antimicrobial group may be intentionally made to be more susceptible to release, and therefore more leachable. This may provide a benefit where it is desirable for a percentage of the antimicrobial groups to be selectively released under certain conditions. However, it is noted that the typical bond between the polymer chain and antimicrobial groups envisioned and enabled herein are covalent bonds that do not leach under standard exposure conditions.

Polymers according to the present invention have the capacity to absorb aqueous liquids such as biological fluids (which are defined to include a liquid having living or dead biologically formed matter, and to include bodily fluids such as blood, urine, menses, etc.). The “capacity to absorb” an aqueous liquid can be measured by the grams of water uptake per gram of absorbent material in a single instance.

One general definition for a “superabsorbent polymer” is that such polymer generally would be capable of absorbing, in a single instance, about 30 to 60 grams of water per gram of polymer. A broader definition could include polymers that absorb less than 30 grams of water per gram of polymer, but that nonetheless have enhanced capacity to absorb water compared to similar materials without such enhanced capacity. Alternately, an “absorbent” as opposed to a “superabsorbent” polymer may be defined as a polymer that has a capacity to absorb aqueous liquids, but which normally will not absorb over 30 times its weight in such liquids.

By “degree of polymerization” is meant the number of monomers that are joined in a single polymer chain. For example, in a preferred embodiment of the invention, the average degree of polymerization is in the range of about 5 to 1,000. In another embodiment, the preferred average degree of polymerization is in the range of about 10 to 500, and in yet another embodiment, the preferred average degree of polymerization is in the range of about 10 to 100.

A “substrate” is defined as a woven or nonwoven, solid, or flexible mass of material upon which the polymers of the invention can be applied and with which such polymers can form covalent bonds. Cellulose products, such as the gauze and other absorbent dressings described in the following paragraphs, are preferred materials to be used as water-insoluble bases when a wound dressing is prepared. The term “substrate” can also include the surfaces of large objects, such as cutting boards, food preparation tables and equipment, surgical room equipment, floor mats, blood transfer storage containers, cast liners, splints, air filters for autos, planes or HVAC systems, military protective garments, face masks, devices for protection against biohazards and biological warfare agents, lumber, meat packaging material, paper currency, powders, including but not limited to mica for cosmetic, antifungal or other applications, and other surfaces in need of a non-leaching antimicrobial property, and the like, onto which is applied the antimicrobial polymeric coating in accordance with the present invention. Apart from cellulose, any material (ceramic, metal, or polymer) with hydroxyl groups or reactive carbon atoms on it's surface can be used as a substrate for the cerium (IV) or other free radical, redox or otherwise catalyzed grafting reaction described in the following paragraphs. The extent of grafting will be dependent on the concentration of surface hydroxyl groups and the concentration of available reactive carbons. Even materials which do not normally contain sufficient surface hydroxyl groups may be used as substrates, as many methods are available for introducing surface hydroxyl groups. These methods generally include hydrolysis or oxidation effected by methods such as heat, plasma-discharge, e-beam, UV, or gamma irradiation, peroxides, acids, ozonolysis, or other methods. It should be noted that methods other than cerium initiated grafting may also be used in the practice of this invention. Thus, for example, not meant to be limiting, a free radical initiator may be used to initiate monomer polymerization. So-called “Azo” initiators, such as VA-057, V-50 and the like, available from Wako Pure Chemical Industries, may be utilized. Other initiators, including but not limited to hydrogen peroxide, sodium persulfate (“SPS”), and the like may also be utilized to advantage according to this invention to initiate polymerization.

The term NIMBUS™ is a coined term used herein as an acronym to refer to a substrate according to the present invention which to refer to Novel Intrinsically Microbicidal Utility Substrates whereby a substrate is derivatized to exhibit antimicrobial efficacy. Thus, polyquaternary amine derivatized substrates exhibit this property. Likewise, a polyquaternary amine derivatized substrate which has been charged with anionic antibiotic, likewise exhibits this property.

It is an advantage of the invention that the wound dressing is capable of absorbing at least 10 times, preferably 20 times, and more preferably greater than 20 times its dry weight of blood, wound exudates, bodily fluid, or 1% saline solution.

It is an aspect of this invention that the inherent non-leachable antimicrobial and/or antiprotease activity is provided by quaternary ammonium moieties which are non-leachably bonded to the wound dressing via covalent chemical bonds.

It is an aspect of this invention that the inherent non-leachable antimicrobial and/or antiprotease activity is provided by polymeric quaternary ammonium molecules which are non-leachably bonded to the wound dressing via covalent chemical bonds.

It is an aspect of this invention that the inherent non-leachable antimicrobial and/or anti-protease activity is provided by polymeric quaternary ammonium molecules, each of which is non-leachably bonded to the wound dressing via a multiplicity of ionic bonds.

It is an aspect of this invention that said polymeric quaternary ammonium molecules form a crosslinked network structure that is capable of forming a hydrogel (a gel structure comprised of said crosslinked network structure and water).

It is an aspect of this invention that said crosslinked network structure is formed via chemical crosslinking of polymeric quaternary ammonium molecules.

It is an aspect of this invention that said crosslinked network structure comprises a polyelectrolyte complex formed between polymeric quaternary ammonium molecules and polymeric anionic molecules.

It is an aspect of this invention that said polymeric anionic molecules comprise carboxymethyl cellulose (CMC), alginate, or polyacrylate.

It is an aspect of this invention that said polymeric quaternary ammonium molecules are covalently bonded to a substrate.

It is an aspect of this invention that said substrate is comprised of cellulose or a cellulose derivative (such as cotton, rayon, carboxymethyl cellulose (CMC), hydroxyethyl cellulose, paper, or woodpulp); a polysaccharide (such as dextran, chitosan, alginate, or starch); a fabric or textile; gauze; fibers; a synthetic polymer; a superabsorbent material; or a protein such as collagen.

It is an aspect of this invention that said polymeric quaternary ammonium molecules comprise poly(dimethyldiallylammonium chloride)—also known as polyDADMAC; quaternary ammonium derivatives of poly(acrylic or methacrylic) acid; poly(vinylbenzyl)trimethylammonium chloride; or compounds generally known as polyquaternium.

It is an aspect of this invention that said inherent non-leachable anti-protease activity is provided by a polymer with a multitude of anionic sites such as carboxymethyl cellulose (CMC), alginate, collagen or polyacrylate.

It is an aspect of this invention that said releasable antimicrobial agent is at least one selected from the group consisting of antibiotics, tetracycline, doxycycline, minocycline and poly(DADMAC).

It is an aspect of this invention that said releasable antiprotease agent is at least one selected from the group consisting of doxycycline, minocycline, tetracyclines, collagen, CMC and poly(DADMAC).

It is an aspect of this invention that said releasable or inherent (non leachable) antimicrobial agent and said releasable or inherent non-leachable anti-protease agent is one and the same, and is at least one selected from the group consisting of CMC, polyDADMAC and doxycycline.

It is an aspect of this invention that said releasable antimicrobial or anti-protease agent exhibits an ionic interaction with said polymeric quaternary ammonium molecules, or with said polymer with a multitude of anionic sites, resulting in ionic stabilization of said agent.

It is an aspect of this invention that said releasable bioactive agents are at least one selected from the group consisting of antibiotics, tetracycline, doxycycline, minocycline, growth factors, epidermal growth factor (EGF), platelet derived growth factor (PDGF), or vascular endothelial growth factor (VEGF), vitamins, nutritive factors, matrix metalloproteinase inhibitors (MMPIs), ilomastat, and steroids.

It is an aspect of the invention that it is an absorbent antimicrobial material comprising a substantially carboxymethylated cellulosic substrate and a plurality of polymeric diallyldimethylammonium chloride molecules non-leachably attached to said substrate, wherein a sufficient amount of said polymeric molecules are attached to said substrate to form a polyelectrolyte network, and wherein said polyelectrolyte network permits the degree of swelling of said material to range from about 10 times up to about 20 times of the dry material, and wherein said polyelectrolyte network diminishes the dissolution of the material upon exposure to aqueous fluids, and wherein said polyelectrolyte network permits the incorporation and release of a bioactive agent in a controlled manner.

It is an aspect of the invention that said polymeric quaternary ammonium molecules impart antimicrobial activity to said material before, during, and after exposure of said material to skin, aqueous biological fluids, bodily fluids, sweat, tears, mucus, urine, menses, blood, or wound exudates.

It is an advantage of the invention that said wound dressing when applied to a mammal reduces the potential for development of infection, inflammation, and malodor.

The present invention provides methods and compositions for an antimicrobial and/or antibacterial composition comprising a substrate over which a non-leaching polymeric coating is covalently bonded. The polymeric coating contains a multitude of quaternary ammonium groups which exert activity against microbes, and also is absorptive of aqueous solutions. A preferred method of fabrication is also described.

It is an aspect of the invention to make a wound dressing that comprises an absorbent, non-leaching antimicrobial surface over a suitable dressing substrate. A typical substrate is cellulose, rayon, or other fibrous mesh, such as a gauze pad. Surprisingly, tests have proved that certain novel forms of polymers on a wound dressing substrate, while not rising to the definition of “superabsorbent” in the parent application, are in fact highly effective at reducing or eliminating the numbers of microbes, including fungi and viruses in addition to a range of bacteria. These embodiments have been shown to be non-leaching. It has also been shown that such materials can be produced on a variety of substrates without significant changes in the physical properties of the substrates such as texture, color, odor, softness, or mechanical strength.

It is an aspect of the invention is to provide a superabsorbent polymer material having antibacterial properties.

It is an aspect of the invention to demonstrate the effectiveness of such a superabsorbent polymer.

Another aspect of the present invention is a composition that comprises a substrate with a covalently bonded superabsorbent polymer surface having antimicrobial properties, which is covered or surrounded by a second substrate to which is covalently bonded another layer of polymer which is not superabsorbent, but which does have a high level of antimicrobial groups. This combination provides both high capacity of liquids absorption, and a high level of antimicrobial activity, while maintaining the feel and handling characteristics of a conventional fabric, particularly in the preferred configuration in which the outermost layer is the non-superabsorbent polymer described herein.

It is an aspect of the invention to include in a dressing or pad according to the present invention an indicator that indicates a condition or the status of the recipient based on some aspect of fluid or other input from the user. For instance, an indicator (such as a color indicator) on a wound dressing may indicate the type of infection or the presence of HIV antibodies, and an indicator (such as a color indicator) on a tampon or like pad may indicate whether or not the user is pregnant, or her HIV status.

It is an aspect of the invention to provide methods and compositions that pertain to antimicrobial surfaces for a variety of supplies and equipment, including a sanitary pad, a tampon, a diaper, a sponge, a sanitary wipe, food preparation surfaces, and other surfaces in need of a non-leaching antimicrobial property.

It is an aspect of the invention to provide, via binding, whether through covalent linkages, ionic interactions, adsorption, or other mechanisms, of significant levels of quaternary amine polymers to powder substrates, including but not limited to mica for inclusion in cosmetic, antifungal, and like compositions whether in a dry or moist form.

It is an aspect of the invention to provide a method for treatment of athlete's foot, (Tinia pedis), jock itch (Tinea cruris), chaffing, and other dermatological conditions in which opportunistic infections or irritations need to be controlled.

It is an aspect of the invention, through high level grafting of quaternary amine polymers according to this disclosure, to modify the properties of powders (including but not limited to micas), fabrics and other substrates to increase the capacity and avidity of dye molecule binding to the cationically treated substrates of this invention.

It is an aspect of the invention to provide compositions and methods whereby a wide variety of biologically and even chemically active compounds are associated with and then released from a polyionic substrate to achieve extended retention and release characteristics in a wide variety of applications from appropriately selected substrates.

### BRIEF DESCRIPTION OF THE DRAWINGS

FIG. 1: Diagrammatic representation of a wound dressing with non-leachable and leachable agents attached.

FIG. 2: Graph illustrating the controlled release of doxycycline by its ability to inhibit bacterial growth following repeated daily inoculation with E. coli and S. aureus.

FIG. 3: Graph of absorbance readings from azocoll assays of polymer treated CMC material, untreated base material, doxycycline loaded treated CMC material, and a commercial dressing.

FIG. 4: Graph of transmittance values from azocoll assays of doxycycline containing superabsorbent polymer (NIMBUS™-SAP doxy), unloaded superabsorbent polymer (NIMBUS™-SAP), untreated Sof-Wick rayon dressing (Sof-Wick), and no substrate (control).

FIG. 5: Graph of cumulative and time point concentration of Epidermal Growth Factor released from NIMBUS™-SAP matrix over time.

FIG. 6: Graph of cumulative and time point concentration of Vitamin C released from NIMBUS™-SAP matrix over time.

FIG. 7: Graph of cumulative and time point concentration of Trolox (water soluble Vitamin E analog) released from NIMBUS™-SAP matrix over time.

### DETAILED DESCRIPTION

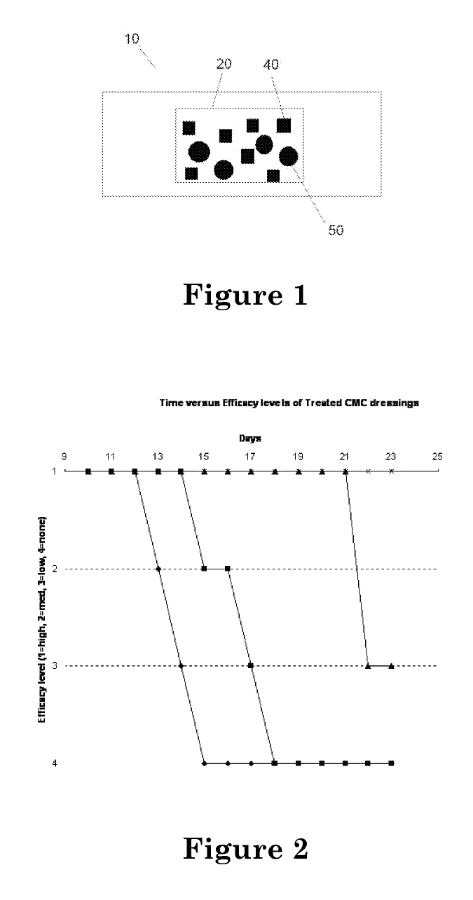

FIG. 1 is a diagrammatic representation of various components present in an embodiment of a wound dressing in accordance with the present invention. Wound dressing 10 is made of cellulosic material which acts as a substrate for non-leachable agents 20, releasable agents 40, and releasable bioactive agents 50. The non-leachable agents 20 comprise one or more kinds of molecule that impart antimicrobial and anti-protease activity into wound dressing 10. Releasable agents 40 and releasable bioactive agents 50 are ionically stabilized within cellulosic material 20, so as to be released from the device in a controlled manner. Releasable agents 40 are comprised of one or more kinds of molecule that impart antimicrobial or anti-protease activity. Releasable bioactive agents 50 aid in wound healing.

Releasable agents are ionically stabilized due to the nature of the polyionic substrates prepared according to various aspects or modifications of this invention. Thus, with respect to wound dressings, a wide variety of antibiotics, proteins, peptides, matrix metalloproteinase inhibitors, analgesics, anti-inflammatory compounds, and the like exhibit net anionic charge at physiological pH, or pH's encountered at a wound site. By contacting these anions with polyquaternary amine functionalized substrates, prepared according to the methods of the present disclosure, the association of these anionic compounds with the substrate is stabilized. Through mass action, displacement of ions and similar mechanisms, the anions associated with the polycationic substrate of this invention are released over time, to exhibit desirable biological effects over a more extended period than would be the case if the biologically active compound were merely absorbed or adsorbed in, on, or to a substantially ionically neutral substrate.

It is an aspect of one exemplary embodiment of the invention that the attachment of polymeric cationic molecules, such as polyquaternary ammonium compounds, to a cellulosic substrate imparts to that substrate an antimicrobial activity effective against a broad range of microorganisms. As used herein, “antimicrobial” refers to the microbicidal or microbistatic properties of a compound, composition, article, or material that enables it to kill, destroy, inactivate, or neutralize a microorganism; or to prevent or reduce the growth, ability to survive, or propagation of a microorganism. As used herein, “microbe” or “microorganism” refers to any organism or combination of organisms able to cause infection, such as bacteria, viruses, protozoa, yeasts, or molds.

A polyelectrolyte network is an arrangement of ionically-charged polymer chains (polyelectrolyte molecules) linked, bonded, or bridged together to form a three dimensional structure. This bonding or bridging of different chains is referred to as “crosslinking”. If the crosslinked molecules are attached to each other by covalent chemical bonds, then the network actually consists of a single molecule. This is what is meant by the term “chemical crosslinking.” The crosslinking may also be achieved by ionic interactions between the chains. For instance, a polymer molecule with multiple cationic sites (a polycation, or cationic polyelectrolyte) may easily dissolve in water or aqueous solutions. The same may be true for a polymer molecule with multiple anionic sites (a polyanion, or anionic polyelectrolyte). The cationic and anionic polymer molecules may interact or associate with each other due to electrostatic attraction of the oppositely charged sites. One cationic molecule may interact with two or more anionic polymer chains, just as an individual anionic molecule may interact with multiple cationic chains. These ionic attractions are called “complexation”. The result is the formation of a three dimensional polyelectrolyte complex network, which is no longer soluble in water or aqueous solutions. Although the ionic crosslinking bonds are not generally as strong or permanent as covalent crosslinking bonds, the sum of many such interactions may give substantially the same effect as covalent crosslinking. The polyelectrolyte complex network or polyelectrolyte network will generally not be soluble in water; however, it is likely to have a great propensity to absorb fluid and swell to form a gel (or hydrogel). An example of a polyelectrolyte network is a hydrogel comprised of covalently crosslinked polyDADMAC chains. The consistency and absorbent capacity of the hydrogel is determined by the extent of covalent crosslinking. An example of a polyelectrolyte complex network is that which is formed by linear (non-crosslinked) polyDADMAC and CMC. The CMC may be linear, branched, or even already a crosslinked polyelectrolyte network. The consistency and absorbent capacity of this hydrogel will depend on the molecular weights and ratio of the two oppositely charged polyelectrolytes. In the case where the CMC is already crosslinked to some extent, addition of polyDADMAC will tend to make the CMC hydrogel less absorbent and less soluble.

It is an aspect of this invention that a covalently crosslinked polycationic network, either alone, or attached to a substrate is used as a component of a wound dressing. This component provides inherent non-leachable antimicrobial and inherent non-leachable anti-protease activity, which is inherent to the polycationic material used. Furthermore, the multitude of cationic sites provided by the polycationic network provides reactive sites for the ionic association and stabilization of releasable antimicrobial, anti-protease, and bioactive agents, which can be released from the wound dressing in a controlled manner. The covalently crosslinked polycationic network is capable of absorbing water or aqueous fluids, resulting in the formation of a hydrogel. In a preferred embodiment, the polycationic network is comprised of poly(DADMAC).

It is an aspect of this invention that a polyelectrolyte complex network formed between a polymeric cationic molecule and a polymeric anionic molecule is used as a component of a wound dressing. This component provides inherent non-leachable antimicrobial and inherent non-leachable anti-protease activity, which is an inherent property of one or both anionic or cationic) polymeric molecules. Furthermore, the multitude of cationic and/or anionic sites provided by the polyelectrolyte complex network provides reactive sites for the ionic association and stabilization of releasable antimicrobial, anti-protease, and bioactive agents, which can be released from the wound dressing in a controlled manner. The polyelectrolyte complex network is capable of absorbing water or aqueous fluids, resulting in the formation of a hydrogel. In a preferred embodiment, the polyelectrolyte complex network is comprised of poly(DADMAC) and CMC.

It is an aspect of one exemplary embodiment of the invention that after attachment of the polymeric cationic or anionic molecules to the substrate, there is a fraction of unbound cationic and/or anionic polymeric molecules remaining within, but not attached to, the treated material. It is an aspect of one exemplary embodiment of the invention that the treated material can be repeatedly rinsed with fluid until substantially all unbound cationic/and or anionic polymer is removed. Alternatively, it is an aspect of one exemplary embodiment of the invention that the unbound fraction can be retained in the treated material to provide additional, leachable, antimicrobial or anti-protease activity. Whether the unbound fraction is rinsed away or retained in the material, the attached fraction of polymeric cationic molecules will serve to form the polyelectrolyte network.

It is an aspect of the invention that the attachment of polymeric cationic molecules to cellulosic substrates forms a polyelectrolyte network with the substrate, and thereby reduces the propensity of the substrate to dissolve when it contacts aqueous fluids. For purposes of illustration of this aspect of the invention, the dissolution of an untreated carboxymethylcellulose (CMC)-based material (e.g. Aquacel®) is relatively fast, with visible dissolution in water occurring within 10-20 minutes under constant agitation and with a significant or complete loss of structure occurring within 4 hours. In contrast, the CMC-based polyelectrolyte complex network material of the current invention does not dissolve in water, or in salt solutions, but maintains its fluid-swollen form for an extended period of time, thus making it a more useful material for wound dressing applications. As used herein, cellulosic means a natural or synthetic material made up, in whole or in part, of cellulose or a cellulose derivative. As used herein, polyelectrolyte network means a network of individual polymeric molecules that interact at nodes of contact formed by charges that coordinate to each other and thereby act as cross-linking points, with the effect of forming smaller and smaller pore size equivalents within the network as the density of the nodes of contact is increased.

It is an aspect of the invention that the attachment of polymeric cationic molecules to anionic cellulosic substrates alters the degree of swelling of the substrate material. The degree of swelling correlates with the amount of polymeric cationic molecules attached and, therefore, the degree of swelling of a treated material can be controlled by varying the amount of polymeric cationic molecules attached to a substrate. The degree of swelling is particularly important for a wound dressing, which must readily absorb wound exudates. Although the degree of swelling of an untreated CMC substrate is difficult to define, because dissolution of untreated CMC substrate begins to occur as soon as it comes into contact with aqueous fluids, in one exemplary embodiment of the current invention, the degree of swelling of a treated substrate can be tailored to be about 10 times to about 20 times its pre-swollen weight.

It is an aspect of the invention that the amount of polymeric cationic molecules attached to the substrate to establish the polyelectrolyte network preferably ranges from about 1% to about 50% of the pre-treated substrate's dry weight; however, the invention is not necessarily limited to this range, as a polyelectrolyte network could be feasibly established with an amount of polymeric cationic molecules that is less than 1% or more than 50% of the pre-treated substrate's dry weight.

It is an aspect of the invention that the controlled-release aspect of the invention is due, at least in part, to at least two factors, each of which can be varied to affect the controlled-release aspect. The first factor is that the density of the polyelectrolyte network structure impedes the transit of agents from the material. The density of the polyelectrolyte network structure can be varied, for example, by varying the amount of cationic polymeric molecules attached to the substrate. The second factor is that a non-uniform charge distribution exists in the polyelectrolyte network due to the attached charge-dense cationic polymeric molecules and this charge-based binding impedes the transit of agents from the material.

It is an aspect of one exemplary embodiment of the invention that when the treated substrate is to be used as a wound dressing material, the material can be formulated to possess protease inhibiting activity in at least two ways. First, CMC material treated with an immobilized stoichiometric excess of polyDADMAC itself has protease binding activity owing to the high density of cationic charge. This means that the material will act in the manner of an anion exchange column. The positively charged surface would act to bind proteases that are predominantly negatively charged at physiological pH of approximately 7. Second, the protease binding activity of the treated CMC material can be augmented by the inclusion of certain controlled-release bioactive agents, such as, but not limited to, the antibiotic doxycycline, which is itself a matrix metalloprotease inhibitor (MMPI). Each of these two properties acting alone, or in concert, can reduce the overall protease activity occurring at the wound site and thereby assist in wound healing.

In one exemplary embodiment of the invention, the substrate is a hydrofiber-based CMC. A hydrofiber-based CMC is a hydrophilic fibrous material that is prepared from cellulosic fibers that are treated with chloroacetic acid in the presence of base in an alcohol solvent, which serves to prevent its gelation. An example of such a hydrofiber-based CMC substrate is ConvaTec® Aquacel®. In other exemplary embodiments of the invention, the substrate is CMC of other forms, such as pre-made gels or powdered CMC. In other exemplary embodiments of the invention, the substrate is a fibrous cellulosic substrate that has first been treated so as to carboxylate the surface, using common methods that are described among other places in U.S. Pat. No. 6,627,785 Edwards et al.

In one exemplary embodiment, a solution of polymerized diallyldimethylammonium chloride is applied to a dry hydrofiber substrate. The substrate material may then be dried, preferably in an oven at, for example, 500-80° C. for about two hours, but more particularly, is dried until the substrate material has been thoroughly dried to a constant weight. Drying at elevated temperatures for a sufficient amount of time for the treated material to reach a constant weight is the preferred way to attach the cationic polymeric molecules to the surface of the substrate, but any method which allows for drying to a constant weight is also appropriate and sufficient, for example, in a vacuum desiccator or a lyophilizer. Drying is a convenient way to fabricate a useful product; however, drying is not essential to the formation of a polyelectrolyte complex network, which may spontaneously form after admixture of the two oppositely charged polyelectrolytes.

It is an aspect of the invention that the cationic polymeric molecules attached to the substrate to form the polyelectrolyte network can be any cationic polymer having a sufficient charge density. It is an aspect of one exemplary embodiment of the invention, that the attached cationic polymer is a polymeric quaternary ammonium compound, which provides additional advantages to the material. First, it provides antimicrobial activity on and within the treated material, a useful property for a material used as a wound dressing. Second, it provides at least some protease binding activity on and within the treated material, a useful property for a material used as a wound dressing.

It is an aspect of the invention, that one, or more than one, controlled-release bioactive agent can be incorporated into the material at the same time that the cationic polymeric molecule is applied to the substrate, or may, alternatively, be applied at any earlier or later time. It is an aspect of the invention that a diverse group of controlled-release bioactive agents can be incorporated into the treated material, including, but not limited to antibiotics (e.g. tetracyclines, including doxycycline), growth factors (e.g. epidermal growth factor (EGF) and platelet derived growth factor (PDGF)), vitamins, nutritive factors, steroids and any charged substance of interest, or MMPIs (e.g. Ilomastat).

Various materials were investigated by the inventors as substrates for the preparation of absorbent dressings containing covalently-bonded, polymeric quaternary ammonium biocidal agent. Among these materials were several commercially-available gauze and surgical sponge products, including several materials manufactured by Johnson & Johnson Company (J&J). J&J's, “NU GAUZE”, General use sponge (referred to in this application as “sub#1”), J&J's “STERILE GAUZE Mirasorb sponge” (herein referred to as “sub#4”), and J&J's “SOFT WICK” dressing sponge (herein referred to as “sub#5”) were all used to prepare working prototypes. All three materials are rayon/cellulose (sub #4 also contains polyester) sheets with non-woven mesh-like structures, and a fiber surface area much greater than traditional woven cotton-fiber gauze. Sub#1 and sub#4 are a single 8″×8″ sheet which is folded into a 4-layer sheet measuring 4″×4″, and both weigh approximately 1.45 to 1.50 grams per sheet. Sub#5 has a denser structure, and is made from a single 12″×8″ sheet folded into a 6-layer sheet measuring 4″×4″, weighing approximately 2.5 grams.

In addition, several types of fabric materials were also used as substrates, including: “Fruit of the Loom” 100% cotton knitted tee-shirt material, “Gerber” 100% cotton bird's-eye weave cloth diaper material, “Cannon” 100% cotton terry wash-cloth material, “Magna” yellow, non-woven wiping cloth (75% rayon, 25% polyester), and “Whirl” cellulose kitchen sponge”; referred to herein as: “subTS”, “subDIA”, “subWC”, “subMag”, and “subCKS” respectively. The scope of this invention is not limited to the use of materials mentioned herein as substrates.

Modification of these substrates to prepare absorbent materials with antimicrobial properties was achieved by immersing the substrates into aqueous solutions of vinyl monomers containing quaternary ammonium groups. Reaction of these monomers with the substrate materials to form graft polymers was catalyzed by ceric ion (Ce⁺⁴), Azo initiators, SPS, or peroxide. A typical modification procedure is detailed in Example 1. Other samples were prepared according to the same basic procedure; however, different substrates, monomers, reaction conditions, washing/drying procedures were used. This data is summarized in Table 1.

Additionally, another aspect of the present invention is the inclusion in a dressing of a hemostatic agent. Hemostatic compounds such as are known to those skilled in the art may be applied to the dressing, either by bonding or preferably added as a separate component that dissolves in blood or wound exudates, and acts to reduce or stop bleeding. In addition, the high positive charge density conferred on substrates due to the application of quaternary amine polymers according to this invention itself provides a surface which facilitates the coagulation cascade.

Furthermore, it will be noted based on the present disclosure that antimicrobial applications of surface treated mica have wide applicability to cosmetics, in which mica is an almost universally included component, with or without titanium dioxide treatment. Inclusion of mica treated according the present disclosure provides a solution, for example, to the situation where a mascara applicator is used, returned to a reservoir bearing adherent microbes which, in the absence of the antimicrobial mica, proliferate in the reservoir. Such proliferation has given rise to increasing levels of concern in the industry and this invention provides a novel, significant and unexpected solution to this long felt need. In addition, the increased dye-binding affinity of substrates, including mica, treated according to the present invention, has applicability to the fabric and cosmetic arts.

The use of cerium(IV) salts as graft polymerization initiators is described above. These salts function by a redox mechanism involving complex formation between the metal ion and the hydroxyl groups on the cellulose substrate. It is known that other metal ions such as V(V), Cr(VI), and Mn(III) function in a similar manner (see P. Nayak and S. Lenka, “Redox Polymerization by Metal Ions”, J. Macromolecular Science, Reviews in Macromolecular Chemistry C19(1), p 83-134 (1980).

Persulfate ion is known as a water-soluble initiator for vinyl polymerizations, but is not widely recognized as a catalyst for graft polymerizations. We have found that sodium persulfate (SPS) functions as a grafting catalyst much in the same manner as the cerium salts used in the parent application (see Examples 3-8, below). There is an advantage for materials prepared from this new catalyst vs. materials prepared using cerium salts, in that the finished materials prepared using SPS show zero discoloration. Samples prepared using cerium catalysts may show a slight off-white, or yellowish discoloration under certain conditions. For most consumer applications it is desirable to have a pure white product. It is possible that materials prepared using the cerium catalyst can contain a small amount of residual cerium, which might be undesirable in the finished product. This is not the case for the SPS system. The by-products of the SPS catalyst are simply sodium ion and sulfate ion, which are completely safe and nontoxic. In general, it is not desirable to have any heavy metal residues in finished medical devices, since some of the heavy metal catalysts described in the above paragraph are rather toxic (chromium, for instance), and could pose hazards for personnel involved in manufacturing, as well as pollution and environmental concerns. An additional benefit of the SPS catalyst is that polymerization may be carried out at room temperature, if desired (see example #4). The grafting reaction using SPS also appears to be quicker than the cerium salt catalyzed reaction. Significant grafting can be achieved in 30 minutes at 60° C. (see example #5), and presumably even quicker at higher temperatures.

The use of peroxydiphosphate and peroxydisulfate as initiators for the graft polymerization of vinyl monomers (but not quaternary monomers) onto silk and wool fibers has been described (see M. Mishra, Graft Copolymerization of Vinyl Monomers onto Silk Fibers, J. Macromolecular Science, Reviews in Macromolecular Chemistry C19(2), p 193-220 (1980). These systems often rely on redox pairs formed by the oxidants (peroxydisulfate or peroxydiphosphate) with reductants such as lithium bromide, or silver nitrate, or are done in the presence of acids such as H₂SO₄. Again, the use of metals such as silver and lithium may lead to undesirable residues in the final products. The use of strong acids is unsuitable for the grafting of cellulose substrates due to severe substrate damage.

Banker reports, in U.S. Pat. No. 5,580,974, the preparation of microfibrillated oxycellulose suitable for use as a carrier in agricultural, cosmetic, and topical and transdermal drug products, and as a binder and disintegrant in the making of tablets, prepared by the oxidation of cellulosic materials with persulfate salts in water, with or without the presence of an aqueous inorganic acid, or in glacial or aqueous acetic acid. No mention of graft polymerization is made.

We have also found (unexpectedly) that other compounds are also capable of catalyzing the grafting of quaternary vinyl monomers onto cellulose. Hydrogen peroxide (HP) is an effective catalyst for this reaction (see Example 9). It is surprising that HP functions in this manner. Although peroxides are generally known to be capable of initiating vinyl polymerizations, it is also well known that oxygen interferes with these processes. Reaction of HP with organic materials liberates elemental oxygen, but this apparently did not prevent grafting. HP is rather useful in that it may be the cleanest catalyst available for preparing these types of graft copolymers. The by-products of HP-catalyzed copolymerization are simply water and oxygen. The by-product of SPS-catalyzed copolymerization is sulfate ion. Sulfate ion is not toxic; however, it is conceivable that its presence in some systems may be undesirable. The HP-catalyzed materials are also very white, with zero discoloration.

Azo compounds such as AIBN (2,2′-azobisisobutyronitrile) are commonly used as initiators for vinyl polymerizations, but are not generally thought of as catalysts for preparation of graft copolymers. We have found, however, that a water-soluble derivative of AIBN (2,2′-Azobis[N-(2-carboxyethyl)-2-methylpropionamidine]tetrahydrate, or VA-057, available from Wako Specialty Chemicals) was a suitable initiator for the graft polymerization of quaternary vinyl monomers onto cellulose (see Example 10). AIBN, which is one of the most commonly used polymerization initiators, is not soluble in water; and thus cannot be used directly in aqueous solutions, as can the various compounds described above. AIBN is soluble in alcohols, however, and thus can possibly be used as an initiator for the graft polymerization of quaternary monomers onto cellulose since the monomers are also soluble in alcohols. It is also likely that AIBN could be used in an emulsion system in order to achieve similar results. Other potentially useful Azo initiators include: (2,2′-Azobis[2-(5-methyl-2-imidazolin-2-yl)propane]dihydrochloride, or VA-041; 2,2′-Azobis{2-methyl-N-[1,1-bis(hydroxymethyl)-2-hydroxyethyl]propionamide, or VA-080; 2,2′-Azobis(2-methylpropionamide)dihydrochloride, or V-50; 2,2′-Azobis(N-cyclohexyl-2-methylpropionamide), or Vam-111; 1,1′-Azobis(cyclohexane-1-carbonitrile); all available from Wako Specialty Chemicals, Inc.; and numerous other similar compounds).

Organic peroxides such as benzoyl peroxide (BPO) are also widely used as polymerization initiators. Just as in the case of AIBN (above), BPO is not water soluble, but it can possibly be used in alcoholic solution in order to graft quaternary vinyl monomers onto cellulose. Other potentially useful peroxide initiators include: (dicumyl peroxide, t-butyl peroxide, methylethylketone peroxide, and a variety of other peroxides, peroxyketals, peroxydicarbonates, and hydroperoxides). These and numerous other potentially useful catalysts are available from a variety of suppliers such as Lucidol-Penwalt, and Akzo.

Combinations of two or more of the initiators described above are also effective (see Example 11). These catalysts can also be used to form crosslinked cellulose-quaternary grafted materials (see example #12).

It should also be noted that the mechanism of action of quaternary compounds is directed towards the cell membrane of the target organism. This process has been described as a mechanical “stabbing” (on a molecular level) which causes rupture of the cell membrane. Thus, it is not possible for pathogenic organisms to develop resistance as observed for most antibiotics.

A further embodiment according to this invention, comprises a wound dressing material capable of controlled or sustained release of a drug such as an antibiotic. However, based on the present disclosure, those skilled in the art will appreciate that the method and compositions disclosed in connection with this embodiment of the invention are not limited to antimicrobials. A variety of other agents, including, for example, matrix metalloproteinase inhibitors, MMPI's, such as Illomostat and its ionic derivatives, may be associated with and released from select polyionic substrates according to this disclosure. Likewise for vitamins, dyes, or other active chemicals such as fragrances. Accordingly, applications of this aspect of the invention are not limited to wound dressings, and include a wide range of applications as specified herein. It should further be noted that the controlled release function of substrates according to this aspect of the invention is in addition to the good antimicrobial properties of polyquaternary amine functionalized substrates as disclosed herein.

In a further aspect according to this embodiment of the invention, an appropriately polyionically derivatized substrate according to this invention is sold as a device, and is loaded with a drug, fragrance, or any of a wide variety of different ionic compounds at the point of sale or use by qualified personnel.

Those skilled in the art will appreciate that many drugs are negatively charged (such as penicillin or vitamin C, as sodium ascorbate). These negatively charged drugs form an ionic bond with a polyquaternary amine derivatized substrate, and prevent them from being washed out quickly from the thus derivatized substrate following ionic interaction between the drug and the polycationic substrate. In comparison, simply coating or infusing a normal untreated substrate (such as cotton or rayon) with drug allows it to be more quickly leached or washed out from the substrate. Complexes formed between the polycationic substrate of this invention and different compounds will have different binding constants, and thus the rate of release will be different. This can be controlled by adjusting the amount of positive charge (graft level), by adjusting the level of drug loading, or by controlling other factors such as surface area or pH. The concept can be extended to positively charged drugs simply by using a negatively charged, i.e. polyanionically derivatized substrate. This is done by grafting acrylic acid monomer onto cellulose, for instance.

In addition to biologically active compounds which are classically considered to be “drugs”, compositions and methods according to this invention can also bind and release more simple ionic compounds such as metal ions (calcium, zinc, silver, rubidium, etc.). Some of these ions are known to be important in wound healing (see, for example, U.S. Pat. No. 6,149,947, hereby incorporated by reference for this purpose). Alternatively, for example, hypochlorite ion may be associated with the derivatized substrate of this invention, and released as an antimicrobial, both for medical or non-medical applications. In yet other embodiments according to this invention, sodium pyrithione is used as a drug to treat fungal skin infections (athlete's foot and dandruff), thereby yielding clothing applications (e.g. socks, undershirts, underwear, derivatized with a polyquaternary ammonium loaded with antifungally effective amounts of sodium pyrithione), foot powder (powder, e.g. “talc” treated with the polyquaternary ammonium polymer according to this invention, and loaded with an antifungally effective amount of sodium pyrithione or another appropriate antifungal). We have demonstrated the enhanced retention of sodium pyrithione (SP) and release over different washing cycles of antimicrobially effective amounts of SP, as compared with complete wash-out in a single cycle of SP using standard substrates.

Illomostat, other MMPIs, and other wound care agents may likewise be associated with and released from the polyionic substrate according to this invention. Both GM1489 molecule (which has a carboxylic acid rather than the hydroxamic acid at the N-terminus of Ilomastat), and the C-terminal carboxylic acid form of Ilomastat (rather than the N-methyl amide in Ilomastat) have a negative charge at physiological pH. Thus, both MMP inhibitors are expected to reversibly bind to, much as do indicator dye molecules and negatively charged antibiotic molecules, as exemplified herein below. Accordingly, wound dressings according to this invention provide sustained release of these potent MMPI molecules. GM1489 has Ki values for MMPs that are almost as good as Ilomastat, and while the Ki values for the C-terminal carboxylic form of Ilomastat are lower, it is still a very acceptable and potent MMPI.

Further, polycationic substrate according to this invention provides sustained release of “PHI or polyhydrated ionogen” active ingredient in Greystone Medical's DerMax dressing. Likewise for proteins such as serine protease inhibitor, alpha-1 protease inhibitor and gelatin (denatured collagen) since these proteins exhibit negative charges at pH 7. Accordingly, per this disclosure, a substrate according to this invention charged with these biologically active compounds provides a dressing with the ability to inhibit MMPs and serine proteases, as is the case for Promogran dressing, except that such a dressing according to this invention would be expected to have better performance for ulcers and bed sores and other wounds caused or exacerbated by matrix metalloproteinases and serine proteinases, because it binds and releases over time inhibitors for both classes of proteases.

In yet a further embodiment of the present invention, a gel, hydrogel, or SAP is utilized as a component of this aspect of the invention. In particular, SAP-polyquaternary ammonium derivatized substrate according to this invention exhibits significant additional advantages. In addition, while grafted polyquaternary amine derivatized substrates are a preferred mode of this invention, simply coated, or otherwise immobilized polyquaternary amine treated substrates are likewise anticipated to operate according to the principles disclosed herein for grafted substrates.

In a yet further embodiment according to this invention, interpenetrating networks (IPNs), or IPNs combined with covalent bonding, is utilized as a variation within the scope of the present invention. Accordingly, coatings are made from polyquat copolymers. For example, a copolymer of TMMC and MMA, soluble in alcohol, but insoluble in water, is permeable or swellable in water. Such a composition is applied from alcohol solution, and does not wash off in water even though it is not covalently bonded. Such a substrate is then charged with polyanionic compounds with desired chemical or biological activities for binding to and then sustrained release from the substrate.

In light of the general disclosure provided herein above, with respect to the manner of practicing this invention, those skilled in the art will appreciate that this disclosure enables the practice of the invention as defined in the attached claims. The following experimental details are provided to ensure a complete written description of this invention, including the best mode thereof. However, it will be appreciated that the scope of this invention should not be construed in terms of the specific examples provided. Rather, the scope of this invention is to be apprehended with reference to the claims appended hereto, in light of the complete description of this inventive method constituted by this entire disclosure.

### EXAMPLES

### Example 1

### Preparation of a Treated CMC Substrate Material

A 4 inch square, hydrofiber CMC wound dressing (i.e. Aquacel®) weighing 1.1 grams and being approximately 24% carboxylated was thoroughly wetted with 20 mL of an aqueous solution of 0.15 wt % polymerized diallyldimethylammonium chloride (total polymer content ˜3%). The wetted dressing was placed in a 60° C. oven on a stainless steel screen mesh and allowed to thoroughly dry until successive weighings indicated no additional considerable loss of weight. To rinse out any unattached polymer, the dressing was submerged in distilled water in a beaker and stirred. The rinsate was poured off and replaced with distilled water repeatedly until the rinsate of a 5 minute soak had the same conductivity as input rinse water, indicating that the rinsate was free of unattached polymer.

### Example 2

### Preparation of a Treated CMC Substrate Material Loaded with Doxycycline

A 4 inch square, hydrofiber CMC wound dressing (i.e. Aquacel®) weighing 1.1 grams and being approximately 24% carboxylated was thoroughly wetted with 20 mL of an aqueous solution of 0.15 wt % polymerized diallyldimethylammonium chloride and 1 wt % doxycycline. The wetted dressing was placed in a 60° C. oven on a stainless steel screen mesh and allowed to thoroughly dry until successive weighings indicated no additional considerable loss of weight. To rinse out any unattached polymer, the dressing was submerged in distilled water in a beaker and stirred. The rinsate was poured off and replaced with distilled water repeatedly until the rinsate of a 5 minute soak had the same conductivity as input rinse water, indicating that the rinsate was free of unattached polymer.

### Example 3

### Demonstration of the Cationic Charge Character of a Treated CMC Substrate Material Using BTB Dye

The anionic pH indicator dye bromothymol blue (BTB) was used to demonstrate the cationic charge character of the CMC substrate material prepared as in Example 1. The treated CMC substrate material was placed into a beaker, saturated with 0.5 wt % BTB dye solution, and allowed to fully absorb the dye for about 5 minutes. The treated CMC substrate material was then rinsed repeatedly with water, until the rinsate no longer visibly contained any BTB dye. After the final rinse, the treated substrate appeared an even, medium to dark blue color. Note that BTB dye can be rinsed from an untreated CMC sample very easily, but that this control is complicated by the relatively rapid dissolution of untreated CMC material in aqueous fluids.

### Example 4

### Microbiological Assay to Verify the Antimicrobial Aspect of Treated CMC Substrate Material

To assay antimicrobial activity, untreated CMC substrate material (control) and CMC substrate materials prepared as in Example 1 (samples) were tested for antimicrobial efficacy by inoculation with 1 mL of a 10⁶ CFU/mL culture of the test bacteria followed by incubation at 37° C. overnight. Samples and controls were then homogenized in phosphate buffered saline and dilutions of the homogenate were used to inoculate bacterial culture plates. The plates were incubated at 37° C. overnight and the colonies were enumerated. Treated sample demonstrated a 3-4 log reduction in the number of bacterial colonies as compared with untreated control.

### Example 5

### Microbiological Assay by Log-Reduction Method to Verify the Antimicrobial Aspect of Treated CMC Substrate Material Loaded with Doxycycline

To assay antimicrobial activity, untreated CMC substrate material (control) and CMC substrate materials prepared as in Example 2 (samples) were tested for antimicrobial efficacy per AATCC method 100-1999: “Antibacterial Finishes on Textile Materials, Assessment of”. The materials were inoculated with 500 μl of a 10⁶ CFU/mL culture of the test bacteria listed in Table E1 below and incubated at 37° C. overnight. The swatches were recovered into transfer solution (with added neutralizing agent), then serially diluted and plated for log reduction quantification as per method instructions.

TABLE E1
Testing of treated CMC materials for microbicidal efficacy

| Sample | Comment | Organism |
|---|---|---|
| Overnight Culture | ATCC # 6538 | SA: Staphylococcus aureus |
| Overnight Culture | ATCC # 15597 | EC: Eschericchia coli |
| Overnight Culture | ATCC # 13880 | SM: Serratia marescens |
| Overnight Culture | ATCC # 15442 | PA: Pseudomonas aeruginosa |
| Overnight Culture | ATCC # BAA-44 | MRSA: methycillin resistant |
| | | Staphylococcus aureus |

| | | | Log red'n | Full Kill * |
|---|---|---|---|---|
| 3.0% wt polymer, 20% wt Doxycycline CMC dressing | | SA | 6.70 | * |
| 3.0% wt polymer, 20% wt Doxycycline CMC dressing | | SA | 6.70 | * |
| 3.0% wt polymer, 20% wt Doxycycline CMC dressing | | SA | 6.70 | * |
| Negative control | Untreated Aquacel CMC dressing | SA | | |
| Negative control | Untreated Aquacel CMC dressing | SA | 5.00E+06 | |
| Negative control | Untreated Aquacel CMC dressing | SA | | |
| 3.0% wt polymer, 20% wt Doxycycline CMC dressing | | EC | 6.70 | * |
| 3.0% wt polymer, 20% wt Doxycycline CMC dressing | | EC | 6.70 | * |
| 3.0% wt polymer, 20% wt Doxycycline CMC dressing | | EC | 6.70 | * |
| Negative control | Untreated Aquacel CMC dressing | EC | | |
| Negative control | Untreated Aquacel CMC dressing | EC | 5.00E+06 | |
| Negative control | Untreated Aquacel CMC dressing | EC | | |
| 3.0% wt polymer, 20% wt Doxycycline CMC dressing | | SM | 7.59 | * |
| 3.0% wt polymer, 20% wt Doxycycline CMC dressing | | SM | 7.59 | * |
| 3.0% wt polymer, 20% wt Doxycycline CMC dressing | | SM | 7.59 | * |
| Negative control | Untreated Aquacel CMC dressing | SM | | |
| Negative control | Untreated Aquacel CMC dressing | SM | 3.86E+07 | |
| Negative control | Untreated Aquacel CMC dressing | SM | | |
| 3.0% wt polymer, 20% wt Doxycycline CMC dressing | | PA | 7.63 | * |
| 3.0% wt polymer, 20% wt Doxycycline CMC dressing | | PA | 7.63 | * |
| 3.0% wt polymer, 20% wt Doxycycline CMC dressing | | PA | 7.63 | * |
| Negative control | Untreated Aquacel CMC dressing | PA | | |
| Negative control | Untreated Aquacel CMC dressing | PA | 4.26E+07 | |
| Negative control | Untreated Aquacel CMC dressing | PA | | |

| | | | |
|---|---|---|---|
| 3.0% wt polymer, 20% wt Doxycycline CMC dressing | MRSA | 6.18 | * |
| 3.0% wt polymer, 20% wt Doxycycline CMC dressing | MRSA | 6.18 | * |
| 3.0% wt polymer, 20% wt Doxycycline CMC dressing | MRSA | 6.18 | * |
| Negative control: prewet CMC | MRSA | | |
| Negative control: prewet CMC | MRSA | 1.50E+06 | |
| Negative control: prewet CMC | MRSA | | |

### Example 6

### Microbiological Assay by Direct Inoculation to Verify the Antimicrobial Aspect of Untreated and Treated CMC Substrate Material with Incorporated Controlled-Release Doxycycline

A treated sample was prepared with doxycycline as described in Example 2. The treated CMC material (sample) that had been concurrently loaded with doxycycline was compared to an Aquacel® dressing (control) that had been loaded with an equal amount (20 ml of 1 w/v % solution) of doxycycline. Sample and control were rinsed with distilled water, and then cut into 15×15 mm squares. These squares were placed onto agar plates and each inoculated with 500 μl of 10⁶ CFU/ml E. coli or S. aureus. The inoculum was placed in the center of the samples, and the samples were incubated at 37° C. for 24 hours. After 24 hours the plates were examined for growth and graded for efficacy level in suppressing bacteria growth on a scale of 1 (high) through 4 (none). This procedure was repeated daily for the periods indicated. The results are shown in FIG. 2, which is a graph of the efficacy level grading over several days. FIG. 2 illustrates the controlled release of doxycycline by its ability to inhibit bacterial growth following repeated daily inoculation with E. coli (indicated by diamond and square points) and S. aureus (indicated by triangle and cross points). Note that in each case the polyDADMAC (PD) treated material (indicated by square and cross points) provided a longer sustained release of doxycycline at microbicidal levels.

### Example 7

### Microbiological Assay to Verify the Antimicrobial Aspect of Treated CMC Substrate Material Loaded with Doxycycline: Time-Kill Assay Per ASTM E-2315 Method Using pseudomonas aeruginosa

Materials prepared as per Example 2 were tested for time to kill as per ASTM E-2315, using pseudomonas aeruginosa (ATCC # 15442). The neutralizing agent utilized was a Letheen broth. Results are provided in Table E2 below, showing that within 2 hours, >99.99% kill was achieved.

TABLE E2
Time kill of pseudomonas aeruginosa (ATCC # 15442) by doxycycline
loaded treated CMC materials, as per ASTM E-2315

| Time | Average log reduction | % kill |
|---|---|---|
| 1 min | 0.24 | 42.7% |
| 10 min | 0.72 | 80.1% |
| 60 min | 2.82 | 99.85% |
| 120 min | 4.49 | 99.997% |

### Example 8

### Protease Inhibition by Treated CMC Material with and without Loaded Doxycycline

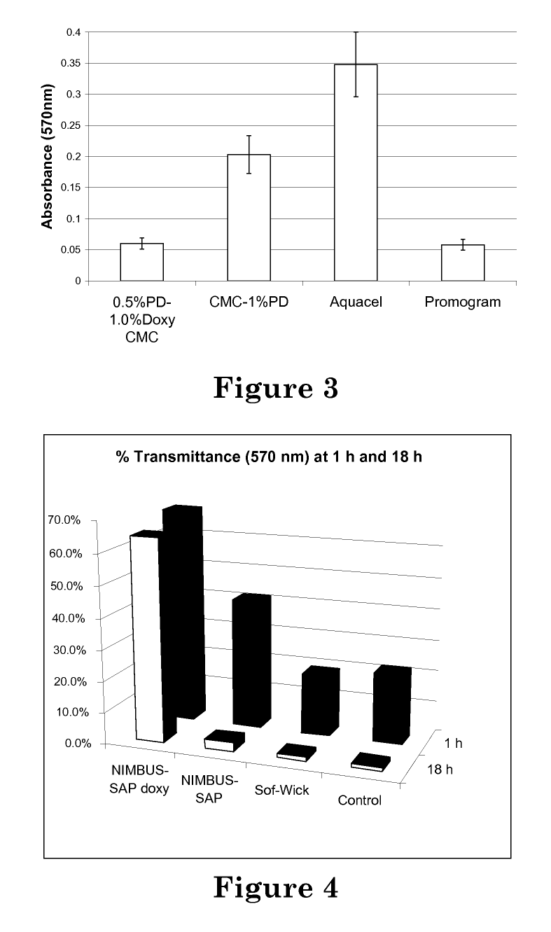

Protease inhibition testing per azocoll assay was performed on several samples and controls. A treated CMC material was prepared as in Example 1, (but treated with a 1% polymer solution to make the total polymer content 20% rather than 3%). Treated CMC with doxycycline was prepared as in Example 2 (but loaded with a total of 10% polymer and 20% doxycycline). These materials were compared to untreated CMC material (Aquacel®), and to commercially available oxidized regenerated cellulose matrix wound dressing (Johnson and Johnson's Promogran™). The materials were evaluated using an azocoll assay and bacterially derived clostridial collagenase. The azocoll assay is familiar to those skilled in the field, and is used to measure collagenase activity. Briefly, insoluble bovine hide (collagen) has dye molecules covalently attached. As the collagen is dissolved by collagenase, dye molecules are released into solution, permitting assessment of collagenase activity by the amount of dye released into solution—with higher absorption measurements indicating more collagenase activity. Lower absorption values indicate that collagenase activity is suppressed: this provides a good measure of protease inhibition. A 20 mg strip of each material was placed into a 1.5 ml microcentrifuge tube with filter insert (0.2 μm filter), and treated with 200 μl of collagenase solution (concentration of 10e⁻⁵), then incubated for 30 min. Following incubation, the samples were centrifuged to extract fluid, and the 50 μl of extract were added to 450 μl of azocoll solution (prepared at a concentration of 10⁻⁵). The azocoll solution was then incubated at 37° C. for 2 h, and transmission measured at 570 nm using a 96-well plate reader for triplicates of each sample. This produced the data shown in FIG. 3. The treated CMC material produced a significant reduction in observed collagenase activity, while the treated CMC material loaded with doxycycline reduced collagenase activity on the same order as the commercial Promogran™ dressing.

### Example 9

### Protease Inhibition by Treated Superabsorbent Polymer Material (NIMBUS™-SAP) with and without Doxycycline Loading

Samples referred to henceforth as “NIMBUS™-SAP” (SAP=superabsorbent polymer) were prepared as per Batich et. al. (U.S. 2002/0177828 A1). This material is a graft copolymer of rayon with DADMAC, wherein the polyDADMAC is covalently crosslinked to form a superabsorbent hydrogel when loaded with water. NIMBUS™-SAP samples were then loaded with doxycycline by aqueous solution loading followed by drying. NIMBUS™-SAP samples were tested for protease inhibition capacity, using azocoll assay to determine the inhibition of collagenase activity. In the case of the NIMBUS™-SAP material the high water absorption capacity requires the use of a higher ratio of inoculum to material, with (500 μl of 10⁻⁵ clostridial collagenase solution per 20 mg sample of NIMBUS™-SAP material). Data was collected at 1 h and at 18 h. The results are shown in FIG. 4 as % transmittance at 570 nm with higher transmittance indicating more protease inhibition by the sample tested. Note the difference between the 1 h and 18 h results for the NIMBUS™-SAP doxycycline sample is minimal, indicating very strong suppression of collagenase activity. Note also that there is a significant difference between the NIMBUS™-SAP material and the control materials (Sof-Wick is rayon dressing material, the labeled control contained no substrate, and represents uncontrolled collagenase activity. The measured data correlated well to the observed color of the samples, as suppression of collagenase activity is inversely proportional to the color of the sample. Samples that are colorless exhibit higher suppression activity. The samples in order of most suppression to least suppression were doxycycline loaded NIMBUS™ superabsorbent polymer, unloaded NIMBUS™ superabsorbent polymer, Sof-Wick rayon substrate, and the no substrate control.

### Example 10

### Incorporation of Growth Factor into NIMBUS™-SAP Material, with Characterized Release

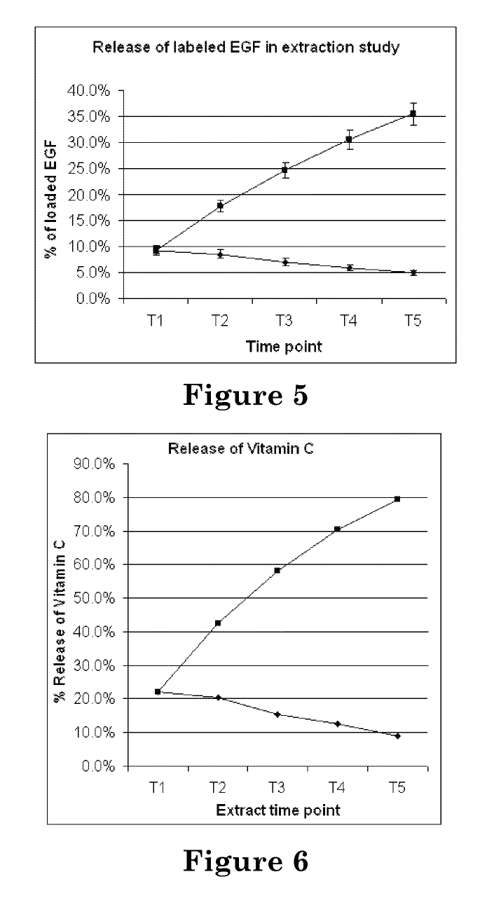

Epidermal Growth Factor (EGF) was labeled with a fluorescein compound, and loaded into a NIMBUS™-SAP superabsorbent polymer matrix, as described for doxycycline loading in Example 9. Release was characterized by performing repeated extraction cycles into PBS, and by UV-visible absorption spectroscopy of the extraction fluid. It was found that EGF concentrations within therapeutic range could be maintained for the duration of the experiment, as illustrated by the graph in FIG. 5.

### Example 11

### Incorporation of Vitamins into NIMBUS™-SAP Material, with Characterized Release

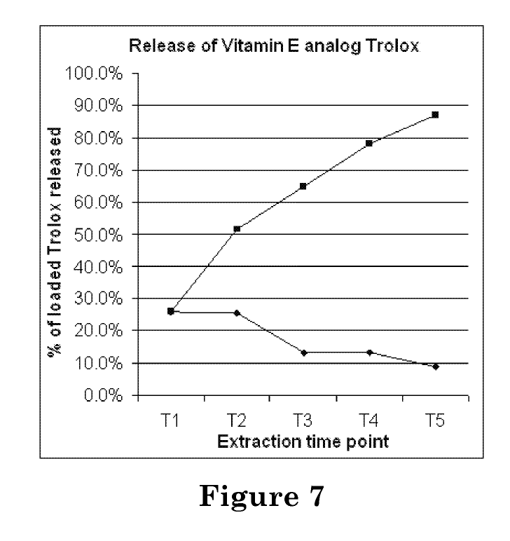

Water soluble vitamins C and vitamin E analog Trolox were loaded into a NIMBUS™-SAP superabsorbent polymer matrix, as described for the material of Example 9. Release was characterized by performing repeated extraction cycles into PBS, and by UV-visible absorption spectroscopy of the extraction fluid. It was found that vitamin concentrations within therapeutic range could be maintained for the duration of the experiment, as is shown in FIG. 6 (Vitamin C) and FIG. 7 (Trolox).

It is easily noticeable that the release of vitamins is faster than that of growth factor. This is reasonable in view of the relative molecular sizes. Thus, a wound dressing can be loaded with excess vitamins to compensate for easier release without adverse effects (physical or economical), allowing the dressing to be designed around the release rate of EGF.

### Example 12

### Incorporation of Multiple Bioactive Components to Form an Advanced Wound Dressings with Tailored Characteristics

The material of Example 9 was used to prepare an advanced wound care dressing with characteristics tailored to suit the wound biochemistry; for this example, a dressing for partial thickness wounds created through chemical vesicant injury. The wound dressing is created by loading the base material, which in one incarnation may be treated CMC as in Examples 1 and 2, and in another incarnation may be a superabsorbent or other material similar to the materials of Examples 9 through 11. Concurrent loading of bioactive agents is easily possible, with concentrations being designed with the release rates of the specific components, as demonstrated in the examples presented. A loading solution composed of growth factor(s), anti-proteases, vitamins and other bioactive agents is added to the dressing, after which the dressing can be dehydrated to the state desired. Full dehydration is the optimal condition to maximize storage life. An embodiment of this dressing is prepared by making an aqueous loading solution containing 0.5% (1 part in 200) of doxycycline, 0.05% (1 part in 2000) each of vitamins C and Trolox: a water soluble vitamin E analog, and 0.01% (100 parts per million) epidermal growth factor (EGF). This solution is loaded into the dressing at 20 times the dry weight of the dressing. The concentrations given provide a therapeutic dose of each ingredient. Extraction testing with 20 mg samples of material, with 600 μl of PBS fluid added and extracted via centrifugation showed that the 5^{th} extract was well above therapeutic levels (as an example, therapeutic levels for growth factors are generally in the range of 1 nM, which equates to 6 ng/ml, whereas the 5^{th} extraction cycle shows concentrations above 12 ng/ml for the conditions given).

### Example 13

### Production of Absorbent Anti-Microbial Compounds

A commercially available surgical sponge rayon/cellulose gauze material (sub#4) was unfolded from its as-received state to give a single layer sheet measuring approximately 8″ by 8″. The sample was then refolded “accordion-style” to give a 6-layer sample measuring approximately 1.33″ by 8″. This was then folded in the same manner to give a 24-layer sample measuring approximately 1.33″ by 2″. This refolding was done so as to provide uniform and maximum surface contact between the substrate and reaction medium, in a small reaction vessel. A solution was prepared by mixing 0.4 grams of ammonium cerium (IV) nitrate (CAN) (Acros Chemical Co. cat #201441000), 25.0 mL [2-(methacryloyloxy)ethyl]trimethyl-ammonium chloride (TMMC) (Aldrich Chemical Company, cat # 40,810-7), and 55 mL of distilled water. This solution was placed into a 250 mL wide-mouth glass container equipped with a screw-cap lid, and argon gas was bubbled vigorously through the solution for 60 seconds. The folded gauze substrate was placed into the solution, and the solution was again sparged with argon for 30 seconds. The container was capped while being flushed with a stream of argon gas. The container was placed into an oven set at 75° C., and gently agitated by hand every 30 minutes for the first two hours, then every hour for the next 4 hours. After a total of 18 hours, the jar was removed from the oven and allowed to cool to room temperature. The sample was removed from the jar, unfolded, and thoroughly washed three times with water, being allowed to soak in water for at least 30 minutes between washings. These sequential washings, also termed rinsings, remove effectively all of the non-polymerized monomer molecules, non-stabilized polymer molecules, and catalyst, such that the final composition is found to not leach its antimicrobial molecules, by routine detection means known and used by those of ordinary skill in the art. By non-stabilized polymer molecules is meant any polymer molecule that has neither formed a covalent bond directly with a binding site of the substrate, nor formed at least one covalent bond with a polymer chain that is covalently bonded (directly or via other polymer chain(s)) to the substrate.

After these rinsings, excess water was removed from the sample by gently squeezing. Further dewatering was accomplished by soaking the sample in 70% isopropanol for 30 minutes. Excess alcohol was removed by gently squeezing the sample, which was then allowed to dry overnight on a paper towel in open air. The sample was then dried in vacuum at room temperature for 18 hours. The sample was allowed to stand in air for 15 minutes before being weighed. The final weight of the sample was measured to be 2.13 grams. The initial weight of the sample before treatment was 1.45 grams. The percent of grafted polymer in the final product was calculated as follows: (2.13−1.45)/2.13×100=31.9%. Some disruption of the fiber packing of the mesh structure was observed, and this resulted in a “fluffier” texture for the treated material.

Preparations of additional samples were performed according to similar procedures using different substrates, antimicrobials and reaction conditions. The reaction conditions and percent grafting data for each sample are summarized in Table E3.

TABLE E3
Ceric ion initiated grafting of gauze substrates

| | | | [Monomer] | | | Total | | | |
|---|---|---|---|---|---|---|---|---|---|
| Sample# | Substrate | Monomer | (mol/L) | [Ce+] (mM) | T (° C.) | Vol. | | % Grafting | |
| 1 | #4 | TMMC | 1 | 11 | 75 | 80 | mL | 12% | |
| 2 | #1 | TMMC | 1.2 | 14 | 75 | 80 | mL | 34% | |
| 3 | #1 (x2) | TMMC | 1.2 | 14 | 75 | 80 | mL | 32% | |
| 4 | #1 | TMMC | 1.2 | 9 | 75 | 80 | mL | 32% | |
| 5 | #1 (x2) | TMMC | 1.2 | 9 | 75 | 80 | mL | 20% | |
| 6 | #1 | TMMC | 0.7 | 14 | 75 | 80 | mL | 28% | |
| 7 | #1 (x2) | TMMC | 0.7 | 14 | 75 | 80 | mL | 27% | |
| 8 | #1 | TMMC | 1 | 11 | 75 | 80 | mL | 37% | |
| 9 | #1 | TMMC | 1 | 11 | 75 | 80 | mL | 37% | |
| 10 | #1 | TMAC | 1.2 | 11 | 75 | 80 | mL | 25% | |
| 11 | #1 | TMAPMC | 1.2 | 10 | 75 | 90 | mL | <1% | |
| 12 | #1 | TMAS | 0.9 | 11 | 75 | 80 | mL | 20% | |
| 13 | #1 | DADMAC | 1.4 | 10 | 75 | 90 | mL | 6% | |
| 14 | #4 (x2) | TMMC | 1.3 | 15 | 90 | 60 | mL | degraded | |
| 15 | #4 (x2) | TMMC | 0.5 | 11 | 90 | 85 | mL | 5% | |
| 16 | #4 (x2) | TMMC | 0.7 | 15 | 90 | 60 | mL | 13% | |
| 17 | #4 | TMMC | 0.7 | 15 | 90 | 60 | mL | 9% | |
| 18 | #1 | TMMC | 1.3 | 15 | 90 | 60 | mL | degraded | |
| 19 | #1 | TMMC | 0.7 | 15 | 90 | 60 | mL | 23% | |
| 20 | #1 | TMMC | 0.4 | 15 | 90 | 60 | mL | 17% | |
| 21 | #4 | TMMC | 1.3 | 15 | 90 | 60 | mL | 26% | |
| 22 | #4 | TMMC | 0.7 | 8 | 75 | 60 | mL | 7% | |
| 23 | #1 | TMMC | 2 | 20 | 75 | 60 | mL | 30% | |
| 24 | #1 | TMMC | 2 | 5 | 75 | 60 | mL | <1% | |
| 25 | #1 | TMMC | 0.7 | 20 | 75 | 60 | mL | 25% | |
| 26 | #1 | TMMC | 0.7 | 7 | 75 | 60 | mL | 15% | |
| 27 | #1 | TMAS | 0.4 | 7 | 60 | 200 | mL | 14% | |
| 28 | #1 | TMAS | 0.2 | 2 | 60 | 200 | mL | 11% | |
| 29 | #1 | TMAS | 0.8 | 10 | 60 | 200 | mL | 19% | |
| 30 | #1 | TMMC | 1 | 11 | 50 | 80 | mL | 44% | |
| 31 | #5 | TMMC | 1 | 11 | 50 | 80 | mL | 48% | |
| 32 | #5 | TMMC | 1 | 11 | 50 | 80 | mL | 48% | |
| 33 | #5 | VBTAC | 0.7 | 78 | 60 | 35 | mL | 15% | |
| 34 | #5 | DADMAC | 2 | 60 | 60 | 60 | mL | 7% | |
| 35 | #5 | VBTAC | 0.4 | 50 | 60 | 37 | mL | 20% | |
| 36 | DIA | TMMC | 0.8 | 11 | 50 | 150 | mL | 12% | |
| 37 | WC | TMMC | 1 | 18 | 65 | 200 | mL | 22% | |
| 38 | MAG | TMMC | 1 | 18 | 65 | 100 | mL | 39% | |
| 39 | CKS | TMMC | 0.8 | 11 | 60 | 150 | mL | 11% | |
| 40 | TS | TMMC | 1 | 15 | 50 | 150 | | 17% | |
| 41 | #5 | TMMC/ | 0.7 | 15 | | | | | |
| | | SR344 | 2.00% | | 60 | 122 | mL | 64% | |
| 42 | #5 | TMMC/ | 0.4 | 15 | | | | | |
| | | SR344 | 2.00% | | 60 | 224 | mL | 79% | |
| 43 | #5 | TMMC | 0.9 | 10 | 75 | 85 | mL | 18% | 30 min. |
| 44 | #5 | TMMC | 0.9 | 10 | 80 | 85 | mL | 21% | 15 min. |
| 45 | #5 (x2) | TMMC | 0.9 | 10 | 55 | 170 | mL | 32% | 2 hours |

NOTES for Table E3:
TMMC = [2-(Methacryloyloxy)ethyl]trimethylammonium chloride (75% solution in water) Aldrich Chemical #40,810-7
TMAS = [2-(Acryloyloxy)ethyl]trimethylammonium methyl sulfate (80% solution in water) Aldrich Chemical #40,811-5
TMAC = [2-(Acryloyloxy)ethyl]trimethylammonium chloride (80% solution in water) Aldrich Chemical #49,614-6
TMAPMC = [3-(Methacryloylamino)propyl]trimethylammonium chloride (50% solution in water) Aldrich Chemical #28,065-8
VBTAC = vinylbenzyltrimethylammonium chloride Acros Chemical #42256
DADMAC = diallyldimethylammonium chloride (65% solution in water) Aldrich Chemical #34,827-9
SR344 = poly(ethylene glycol)diacrylate Sartomer Company # SR344
All procedures were performed in 500 mL or 250 mL screw-cap glass jars overnight (approximately 18 hours), except for samples #43-45 which were reacted for indicated times.

The samples prepared as shown in Table E3 indicated that high-yield grafting of vinyl monomers containing quaternary ammonium groups onto various textile substrate materials can be achieved under rather mild conditions. The appearance of the prepared biocidal absorbent dressings generally was identical to that of the starting material. Parameters such as mechanical strength, color, softness, and texture were found to be sufficient and acceptable for use in the various applications mentioned above. For instance, the materials based on medical dressings were soft, white, odorless, and absorbent. Storage of these materials for several months yielded no observable physical changes. The same holds true for heat treatments of 75° C. for several hours (this is not meant to be a limiting condition).

It should be noted that although these examples demonstrate modification of textile fabrics already in finished form, it is also within the scope of this invention to achieve the grafting modification at the raw materials stage. Threads, yarns, filaments, lints, pulps, as well as other raw forms may be modified and then fabricated into useful materials or fabrics (woven or nonwoven) by weaving, knitting, spinning, or other forming methods such as, spunbonding, melt blowing, laminations thereof, hydroentanglement, wet or dry forming and bonding, etc.

Grafting yields were found to be reproducible with constant formulation and reaction conditions. Samples were thoroughly washed to remove any residues such as unreacted monomer or homopolymer. Degree of grafting was calculated based on the weight of the starting material and the final dried weight of the grafted material. The calculated values of percent grafting are subject to a certain degree of error based upon the fact that the materials appear to contain a small amount of adsorbed water due to exposure to the laboratory atmosphere. This is true even for the untreated starting materials which were generally found to show a reversible weight loss of approximately 5 to 7% after being dried in a 60° C. oven for 30 minutes. Another potential source of error is the possibility of the presence of other counterions besides chloride (bromide, or nitrate, for instance). Experiments were conducted to correlate the weight of treated samples after washing with excess salt solutions of various composition. Related to this is the well-known observation that quaternary ammonium compounds strongly bind sodium fluorescein dye to form a colored complex. Various samples from Table E3 were tested by immersing them in a concentrated (5%) solution of sodium fluorescein, followed by drying, and then thorough washing in water. Untreated fabrics did not retain any color after this treatment; however, all treated materials showed a pronounced color which ranged from light orange to dark brown, depending on the quaternary ammonium content. In one case (a sample identical to that of Sample #31), the fluorescein treated sample showed a weight gain of 27%. Further analysis on this sample for % nitrogen and % chloride was conducted by an independent laboratory (Galbraith Laboratories, Inc., Knoxville, Tenn.). The results (2.62% N and 6.83% Cl) indicate a slightly lower level than as calculated gravimetrically. This is likely due to the reasons described above. An exact control of % grafting is not a requirement of this invention. As described in the testing presented below, the antimicrobial activity of these materials is functional over a wide range of compositions.

The materials described by Sample #1 through Sample #40 are graft copolymers in which the quaternary ammonium polymeric grafts have a linear structure. These highly charged linear chains would be water-soluble if they were not tethered at one end to a cellulose substrate. Thus, the materials are capable of absorbing and holding water. Selected materials were tested for their ability to absorb and retain water. For instance, a 2.22 gram sample of the material of Sample #2 was found to retain 12.68 times its own weight of water when placed in a funnel and completely saturated. The samples prepared in Sample #41 and Sample #42 were found to retain water at 38 and 66 times their own weight, respectively. These two samples were prepared using a combination of monofunctional quaternary monomer, and a difunctional non-quaternary cross linking agent. The cross linking agent causes the grafted polymer chains to become branched, and also allows individual chains to form chemical bonds with each other that result in network formation. Once swollen with water, the polymer network becomes a slippery gel material. The absorbent biocidal materials produced with and without cross linking agent have similar chemical and antimicrobial properties. Although the materials prepared using cross linking agents have extremely high absorbing capacity, they do tend to become rather slippery when wet.

This slippery property may be undesirable in some applications, particularly where this is the exposed surface. However, the two different variations may be utilized in conjunction with each other. For instance, the material of Sample #35 may be used as a shell or barrier material around the material of Sample #42. This would result in a bandage material having a superabsorbent compound interiorly to provide absorptive capacity, having inherent antimicrobial properties throughout, and having superior antimicrobial properties on the exterior (where a polymer having antimicrobial properties that are demonstrated superior to a polymer with superabsorptive capacity is employed in the outer location).

### Example 14

### Testing of Antimicrobial Activity

All biological testing was performed by an independent testing laboratory (Biological Consulting Services of North Florida, Incorporated, Gainesville, Fla.). The first set of antimicrobial activity tests was performed using the absorbent antimicrobial material of Sample # 21. The grafting yield for this sample was 26%. An untreated, unwashed sample of as-received sub#4 was used as a control. A sample of sub#1 treated with a siloxane based quaternary formulation (TMS, or 3-(trimethoxysilyl)-propyloctadecyldimethyl ammonium chloride) was also tested (sample # 1122F). This sample contained approximately 9% quaternary siloxane which was applied from methanol solution. Based on a series of experiments with this quaternary siloxane, this is the maximum level which could be successfully applied to the substrate material. It was later found that the applied siloxane quaternary treatment was unstable, as evidenced by significant weight loss after washing the treated material after 30 days storage. This level is also higher than is typically achieved in antimicrobial treatments of similar substrates using commercial TMS products. It should also be noted that there were difficulties during the testing due to the hydrophobic (water-repellent) nature of the siloxane-treated material. Such properties are not desirable in a product designed specifically to be highly absorbent.

In a modification of the AATCC-100 antimicrobial test protocol, gauze material from these three samples was aseptically cut into squares weighing 0.1±0.05 grams. This corresponds to a 1″×1″ four-layer section. Each square was then individually placed in a sterile 15-mm petri dish and covered. One-milliliter tryptic soy broth suspension containing 10⁶-cfu/ml mid-log phase E. coli (ATCC 15597) or S. aureus (ATCC 12600) was added to each gauze section. The plates were then incubated overnight at 37° C. Following incubation, the material was aseptically placed into 50-mL conical centrifuge tubes. Twenty-five milliliters of sterile phosphate buffered saline was then added to each tube. The tubes were shaken on a rotary shaker (Red Rotor PR70/75, Hoofer Scientific, CA) for 30 minutes. The eluant was then diluted accordingly and enumerated by aseptically spread plating onto Tryptic Soy Agar (TSA) plates. The plates were incubated overnight at 37° C. All gauze samples were processed in triplicates. The results of this testing are summarized in Table E4.

TABLE E4
Results of antimicrobial activity testing.

| | cfu/mL | |
|---|---|---|
| Sample | Staphylococcus aureus | Escherichia coli |
| | 1.3 × 10⁶ | 6.1 × 10⁶ |
| Sub#4 (control) | 4.6 × 10⁵ | 2.4 × 10⁶ |
| | 8.0 × 10⁵ | 1.5 × 10⁶ |
| | <10 | <10 |
| Material of Sample #21 | <10 | <10 |
| | <10 | <10 |
| | <10 | 1.4 × 10⁴ |
| TMS siloxane Material | 20 | 2.3 × 10⁴ |
| | 170 | 4.3 × 10⁴ |

The results of this experiment are rather self-explanatory, and indicate that the material of Sample #21 was able to kill greater than 99.999% of both organisms. The siloxane-based quaternary ammonium sample (DC5700) was fairly effective on S. aureus, but only slightly effective on E. coli.

Further testing was carried out using the materials of Sample # 9. A freshly-prepared sample of sub#1 treated with TMS siloxane quaternary ammonium (8%) was also tested, along with a washed untreated sub#1 control. In this experiment, freshly-prepared bacterial cultures containing additional TSB growth medium were used. The samples were treated as before. In addition, a second set of samples was reinoculated with additional bacterial culture after the first day of incubation, and allowed to incubate for an additional day. Data from these experiments is presented in Tables E5 and E6.

TABLE E5
Colony forming units (cfu) of 4 layer gauze strips cut into one inch²
sections following inoculation with bacteria and overnight incubation.

| | cfu/mL | |
|---|---|---|
| Sample | Staphylococcus aureus | Escherichia coli |
| (Control) | 5.2 × 10⁷ | 8.7 × 10⁷ |
| (Sub#1 washed) | 2.1 × 10⁷ | 4.6 × 10⁷ |
| | 9.4 × 10⁷ | 5.4 × 10⁷ |
| TMS siloxane quat | 1.2 × 10⁶ | 8.8 × 10⁶ |
| (8% on Sub#1) | 9.1 × 10⁶ | 1.3 × 10⁷ |
| | 5.9 × 10⁶ | 7.0 × 10⁶ |
| Material of Sample #9) | 8.9 × 10¹ | 6.6 × 10¹ |
| (37% TMMC on sub#1) | 3.7 × 10¹ | 3.6 × 10¹ |
| | 3.3 × 10¹ | 9.0 × 10⁰ |

TABLE E6
Colony forming units (cfu) of 0.1-gram gauze strips following
inoculation with the indicated bacteria, overnight incubation,
re-inoculation, and overnight incubation.

| | cfu/mL | |
|---|---|---|
| Sample | Staphylococcus aureus | Escherichia coli |
| (Control) | 5.6 × 10⁸ | 3.9 × 10⁸ |
| (Sub#1 washed) | 2.6 × 10⁸ | 3.8 × 10⁸ |
| | 4.2 × 10⁸ | 1.9 × 10⁸ |
| TMS siloxane quat | 2.1 × 10⁶ | 2.2 × 10⁸ |
| (8% on Sub#1) | 1.8 × 10⁶ | 1.8 × 10⁸ |
| | 8.0 × 10⁵ | 2.7 × 10⁸ |
| Material of Sample #9 | 3.4 × 10¹ | 6.7 × 10² |
| (37% TMMC on sub#1) | 3.8 × 10² | 7.2 × 10¹ |
| | 9.1 × 10¹ | 5.9 × 10¹ |

Again, the results of this experiment are self-explanatory. The siloxane-based quaternary ammonium did not show significant antibacterial activity, whereas the TMMC-grafted material did.

In another experiment, the antimicrobial effectiveness of several materials was tested in the presence of a high concentration of bodily fluids, as expected to occur in a heavily draining wound, for instance. The procedure was similar to that described above, except that the bacterial levels were higher (10⁸ cfu/mL), and the inoculation mixture contained 50/50 newborn calf serum and TSB. The samples tested in this experiment were those of Samples #30 and 31. In addition, a sample of siloxane quaternary ammonium-treated knitted cotton material was obtained from a commercial supplier (Aegis). The results are presented in Table E7.

TABLE E7
Testing of biocidal absorbent materials in presence of 50%
calf blood serum

| | cfu/mL | |
|---|---|---|
| Sample | Staphylococcus aureus | Escherichia coli |
| Control | 5.9 × 10⁷ | 2.7 × 10⁷ |
| “Sub#5” | 6.3 × 10⁷ | 1.9 × 10⁷ |
| J&J gauze | 7.1 × 10⁷ | 9.8 × 10⁶ |
| Siloxane quat on | 1.8 × 10⁷ | 1.2 × 10⁶ |
| Cotton fabric | 3.5 × 10⁷ | 9.5 × 10⁵ |
| | 1.5 × 10⁷ | 7.0 × 10⁶ |
| Material of Sample 30 | 1.0 × 10⁴ | <1.0 × 10⁰ |
| TMMC quat | 1.2 × 10⁴ | 5.0 × 10⁰ |
| ~44% graft | 9.7 × 10³ | <1.0 × 10⁰ |
| Material of Sample 31 | 2.4 × 10⁴ | 3.9 × 10² |
| TMMC quat | 3.2 × 10⁴ | 6.0 × 10⁰ |
| ~48% graft | 1.2 × 10⁵ | 1.0 × 10⁰ |

As can be seen from the data in Table E7, the siloxane based quaternary treated material showed almost zero effectiveness. The TMMC-grafted material was extremely effective against e-coli, even in the presence of high concentrations of bodily fluids. The high serum protein concentration appeared to mask the effectiveness of the TMMC-grafted material to some extent; however, the levels of serum which were used in this experiment were quite challenging. Generally, in these types of experiments much lower serum levels are used (10 to 20%).

It is an aim of this invention to provide an absorbent antimicrobial material which does not leach or elute any soluble antimicrobial agent. In order to verify this, material of sample #31 (Table E3) was extraction tested under a range of pH conditions, and also in the presence of blood serum. In addition, a commercially available antimicrobial dressing was also tested under identical conditions. The commercially available antimicrobial dressing is “Kerlix-A.M.D. Antimicrobial Super Sponges”, manufactured by Kendall Tyco Healthcare Group (active ingredient 0.2% Polyhexamethylene Biguanide). The following procedure was used: Approximately, a one square inch section of each bandage material was placed in a 50-mL sterile polypropylene tube. Twenty-five milliliters of phosphate buffered saline at pH 5.0, pH 7.0, pH 7.0 supplemented with 10% fetal bovine serum (FBS), or pH 9.0 was added to each tube. Each sample was processed in triplicates to assure reproducibility. pH values were adjusted using 0.1 N NaOH or HCl. The tubes ware then placed on a rotary shaker (Red Rotor PR70/75, Hoofer Scientific, CA) for and agitated mildly (40 rotations/min) for 16 hours. Tryptic Soy Agar (TSA) (Difco Laboratory, Detroit, Mich.) petri dishes were inoculated with a continuous lawn of either E. coli (ATCC 15597) or S. aureus (ATCC 12600) and the plates were divided into four sections. Twenty microliters of the soaked gauze aqueous extract was then placed onto the labeled sections of the bacteria inoculated plates. The plates were then covered and incubated at 37° C. for 18 hours. The plates were then visibly inspected for growth suppression at areas of inoculation. The results are presented in Table E8.

TABLE E8
Anti Microbial release test of supplied gauze material after soaking in
Phosphate buffered saline (PBS) for 16 hours at various pH values

| | Effect of Gauze Extract on | |
|---|---|---|
| | Bacterial Growth | |
| Sample | Staphylococcus aureus | Escherichia coli |
| pH 5.0 | No Inhibition | No Inhibition |
| Material of Sample #31 | No Inhibition | No Inhibition |
| | No Inhibition | No Inhibition |
| pH 7.0 | No Inhibition | No Inhibition |
| Material of Sample #31 | No Inhibition | No Inhibition |
| | No Inhibition | No Inhibition |
| PH 7.0 | No Inhibition | No Inhibition |
| Material of Sample #31 | No Inhibition | No Inhibition |
| with 10% FBS | No Inhibition | No Inhibition |
| PH 9.0 | No Inhibition | No Inhibition |
| Material of Sample #31 | No Inhibition | No Inhibition |
| | No Inhibition | No Inhibition |
| PH 5.0 | Inhibition | No Inhibition |
| (Kerlix AMD) | Inhibition | No Inhibition |
| | Inhibition | No Inhibition |
| PH 7.0 | Inhibition | No Inhibition |
| (Kerlix AMD) | Inhibition | No Inhibition |
| | Inhibition | No Inhibition |
| PH 7.0 | Inhibition | No Inhibition |
| With 10% FBS | Inhibition | No Inhibition |
| (Kerlix AMD) | Inhibition | No Inhibition |
| PH 9.0 | Inhibition | No Inhibition |
| (Kerlix AMD) | Inhibition | No Inhibition |
| | Inhibition | No Inhibition |

As seen from the results listed in Table E8, the material of sample #31 did not leach or release any antimicrobial agent under any of the conditions tested; however, the commercial antimicrobial dressing, Kerlix AMD, was found to release antimicrobial agent toxic to S. aureus under all testing conditions. Such leaching of active agent may have an undesirable effect on wound healing, and also cause decreased antimicrobial effectiveness of the dressing.

Further antimicrobial testing in the presence of 10% blood serum was performed using additional organisms as described below. These included a number of common pathogenic bacteria, as well as at least one fungal species. The material of Sample #32 was tested, and untreated sub#5 was used as a control. The gauze material was aseptically cut into approximately one inch square sections. Sub#5 gauze sample consisted of material in four layers and the material of Sample #32 consisted of two layers. Both types of samples weighed approximately 0.1 gram. Each sample section was individually placed in a sterile 100×15-mm petri dish and covered. Escherichia coli (ATCC 15597), Staphylococcus aureus (ATCC 12600), Klebsiella pneumoniae (ATCC 13883), Pseudomonas aeruginosa (ATCC 51447), Proteus vulgaris (ATCC 13115), Serratia marcescens (ATCC13880), Enterococcus faecalis (ATCC 19433), and Enterobacter aerogenes (ATCC 13048) were grown in twenty five milliliters of tryptic soy broth (TSB) (Difco Laboratory, Detroit, Mich.) for 16 hours at 37° C. Each bacterial culture was then diluted in Fresh TSB or PBS containing 10% Fetal Bovine Serum (Sigma, St. Louis, Mo.) to a final concentration of approximately 10⁵-cfu/mL. One milliliter of each bacterial suspension was added to each gauze section. Each section was inoculated with only one bacterial species. All gauze samples were inoculated in triplicates. The petri dish containing the inoculated sample was then incubated for 18 hours at 37° C. in 95% humidity. Following incubation, the gauze material was aseptically placed into 50-mL conical centrifuge tubes. Twenty-five milliliters of sterile phosphate buffered saline (PBS) was then added to each tube. The tubes were shaken on a rotary shaker (Red Rotor PR70/75, Hoofer Scientific, CA) for 30 minutes. The eluant was then serially diluted. Tenfold dilutions were performed by the addition of 0.3-ML of sample to 2.7-mL of sterile PBS. Aliquots of each dilution or of the original undiluted sample were then aseptically spread plated onto Tryptic Soy Agar (TSA) (Difco Laboratory, Detroit, Mich.) plates. The plates were incubated for 18 hours at 37° C. The colonies on the respective plates were counted and concentrations were determined. The fungus Candida albicans was used in the same procedure outlined above, except incubation times were doubled and Sabouraud Dextrose Broth and agar (Difco Laboratory, Detroit, Mich.) were used instead of TSB and TSA, respectively. The results are summarized in Table E9. The reported bacterial levels are diluted by a factor of 25× versus the level present in the actual gauze samples.

TABLE E9
Antimicrobial activity results for various organisms.

Sample

| Organism | Sub 5* (control) | Material of Sample 32 |
|---|---|---|
| S. aureus | 5.9 × 10⁶ | <2.0 × 10⁰ |
| S. aureus | 6.3 × 10⁷ | <2.0 × 10⁰ |
| S. aureus | 7.1 × 10⁷ | <2.0 × 10⁰ |
| E. coli | 1.7 × 10⁶ | <2.0 × 10⁰ |
| E. coli | 1.9 × 10⁶ | <2.0 × 10⁰ |
| E. coli | 2.4 × 10⁶ | <2.0 × 10⁰ |
| K. pneumoniae | 1.8 × 10⁶ | <2.0 × 10⁰ |
| K. pneumoniae | 1.4 × 10⁶ | <2.0 × 10⁰ |
| K. pneumoniae | 3.7 × 10⁶ | <2.0 × 10⁰ |
| P. aeruginosa | 2.1 × 10⁷ | <2.0 × 10⁰ |
| P. aeruginosa | 3.9 × 10⁷ | <2.0 × 10⁰ |
| P. aeruginosa | 4.3 × 10⁷ | <2.0 × 10⁰ |
| P. vulgaris | 2.8 × 10⁶ | <2.0 × 10⁰ |
| P. vulgaris | 1.1 × 10⁷ | <2.0 × 10⁰ |
| P. vulgaris | 3.7 × 10⁶ | <2.0 × 10⁰ |
| S. marcescens | 6.7 × 10⁷ | <2.0 × 10⁰ |
| S. marcescens | 7.3 × 10⁷ | <2.0 × 10⁰ |
| S. marcescens | 8.7 × 10⁷ | <2.0 × 10⁰ |
| E. faecalis | 3.8 × 10⁶ | <2.0 × 10⁰ |
| E. faecalis | 1.7 × 10⁶ | <2.0 × 10⁰ |
| E. faecalis | 2.9 × 10⁶ | <2.0 × 10⁰ |
| E. aerogenes | 1.1 × 10⁷ | <2.0 × 10⁰ |
| E. aerogenes | 3.3 × 10⁷ | <2.0 × 10⁰ |
| E. aerogenes | 2.9 × 10⁷ | <2.0 × 10⁰ |
| C. albicans | 5.9 × 10⁵ | 2.0 × 10⁰ |
| C. albicans | 7.2 × 10⁵ | 4.0 × 10⁰ |
| C. albicans | 1.2 × 10⁶ | 5.0 × 10⁰ |

*values represent cfu/mL of the 25-mL PBS solution used to elute the microorganisms from the gauze sections.

The results presented in Table E9 indicate significant antimicrobial activity for the TMMC-grafted material against a variety of organisms. Further testing of this material was conducted using several bacteriophages. Bacteriophages are viral organisms which infect a particular bacterial host. In this method, the antimicrobial material is inoculated with the viral agent and then allowed to incubate for a specified period. The amount of viable viral organism is then determined on the basis of remaining ability to infect the host bacteria. Samples were aseptically cut into approximately one inch² square sections. Sub#5 gauze sample consisted of material in four layers and the material of Sample #32 consisted of material in two layers. Each sample weighed approximately 0.1 g. Each sample section was individually placed in a sterile 100×15-mm petri dish and covered. Stocks of the following bacteriophages, MS2 (ATCC 15597-B1), φX-174 (ATCC 13706-B1), and PRD-1 were added to 10 mL of TSB or PBS containing 10% Fetal Bovine Serum (Sigma, St. Louis, Mo.) to a final concentration of approximately 10⁶-cfu/mL. One milliliter of each bacterial suspension was added to each gauze section. All gauze samples were inoculated in triplicates. The petri dish containing the inoculated sample was then incubated for 18 hours at 37° C. in 100% humidity. Following incubation, the gauze material was aseptically placed into 50-mL conical centrifuge tubes. Twenty-five milliliters of sterile phosphate buffered saline (PBS) was then added to each tube. The tubes were shaken on a rotary shaker (Red Rotor PR70/75, Hoofer Scientific, CA) for 30 minutes. The eluant was then serially diluted. Tenfold dilutions were performed by the addition of 0.3-ML of sample to 2.7-mL of sterile PBS. Phages were assayed as plaque-forming units (pfu) using their respective hosts (MS2 (ATCC 15597-B1), Escherichia coli C-3000 (ATCC 15597); φX-174 (ATCC 13706-B1), E. coli (ATCC 13706); and PRD-1, Salmonella typhimurium (ATCC 19585)). The soft-agar overlay method (Snustad, S. A. and D. S. Dean, 1971, “Genetic Experiments with Bacterial Viruses”. W.H. Freeman and Co., San Francisco) was used for enumerating the phages. The results are presented in Table 10.

TABLE E10
Testing of absorbent antimicrobial material against viral agents.

Sample

| Bacteriophage | Sub #5 (control)* | Material of Sample #32 |
|---|---|---|
| MS-2 | 3.3 × 10⁴ | <2.0 × 10⁰ |
| MS-2 | 4.1 × 10⁴ | <2.0 × 10⁰ |
| MS-2 | 2.3 × 10⁴ | <2.0 × 10⁰ |
| PRD1 | 1.7 × 10⁵ | 1.2 × 10² |
| PRD1 | 7.9 × 10⁴ | 1.5 × 10² |
| PRD1 | 8.8 × 10⁴ | 1.7 × 10² |
| φX-174 | 8.7 × 10³ | 2.4 × 10³ |
| φX-174 | 1.2 × 10⁴ | 1.1 × 10³ |
| φX-174 | 9.0 × 10³ | 1.7 × 10³ |

*values represent pfu/mL of the 25-mL PBS solution used to elute the microorganisms from the gauze sections.

As seen from the results in Table E10, the absorbent antimicrobial material has significant effectiveness against viral pathogens, as evidenced by reduction or loss of bacteriophage activity in the treated Sample #32. These results, in combination with the results regarding bacterial and fungal organisms, indicate a relatively broad antimicrobial potential for compositions of the invention.

In additional testing, several absorbent antimicrobial dressing materials not based on acrylate materials were studied. These tests included the materials of Samples #33, with VBTAC, and #34, with DADMAC. The material was aseptically cut into two layer square sections of approximately one inch². Each square was individually placed in a sterile 100×15-mm petri dish and covered. E. coli (ATCC 15597) and S. aureus (ATCC 12600) were grown in twenty five milliliters of tryptic soy broth (TSB) (Difco Laboratory, Detroit, Mich.) for 16 hours at 37° C. Each bacterial culture was then diluted in Fresh TSB or PBS containing 10% Fetal Bovine Serum (Sigma, St. Louis, Mo.) to a final concentration of approximately 10⁵-cfu/mL. One milliliter of each bacterial suspension was added to each gauze section. Each section was inoculated with only one bacterial species. All gauze samples were inoculated in triplicates. The petri dish containing the inoculated sample was then incubated for 16 hours at 37° C. in 95% humidity. Following incubation, the gauze material was aseptically placed into 50-mL conical centrifuge tubes. Twenty-five milliliters of sterile phosphate buffered saline (PBS) was then added to each tube. The tubes were shaken on a rotary shaker (Red Rotor PR70/75, Hoofer Scientific, CA) for 30 minutes. The eluant was then serially diluted. Tenfold dilutions were performed by the addition of 0.3-ML of sample to 2.7-mL of sterile PBS. Aliquots of each dilution or of the original undiluted sample were then aseptically spread plated onto Tryptic Soy Agar (TSA) (Difco Laboratory, Detroit, MI) plates. The plates were incubated for 18 hours at 37° C. The colonies on the respective plates were counted and concentrations were determined. The results are summarized in Table E11.

TABLE E11
Colony forming units (cfu) present in the PBS eluant (25-mL) of the
indicated gauze sections (1-inch²) following their inoculation
with bacteria and overnight incubation:

| | cfu/mL of the PBS eluant | |
|---|---|---|
| Sample | Staphylococcus aureus | Escherichia coli |
| SUB 5 | 7.6 × 10⁶ | 1.6 × 10⁷ |
| (CONTROL) | 6.9 × 10⁶ | 2.9 × 10⁷ |
| | 5.8 × 10⁶ | 1.3 × 10⁷ |
| Material of Sample #33 | <1.0 × 10⁰ | <1.0 × 10⁰ |
| 15% VBTAC graft | <1.0 × 10⁰ | <1.0 × 10⁰ |
| | <1.0 × 10⁰ | <1.0 × 10⁰ |
| Material of Sample #34 | 3.0 × 10⁰ | <1.0 × 10⁰ |
| 7% DADMAC graft | 4.0 × 10⁰ | <1.0 × 10⁰ |
| | <1.0 × 10⁰ | <1.0 × 10⁰ |

As shown in Table E11, the material with grafted quaternary ammonium polymer showed significant antimicrobial activity, even in the presence of 10% blood serum.

Additional verification of the nonleaching nature of the subject materials was obtained by Kirby-Bauer zone of inhibition tests. Sample #32 was used in this experiment, along with a control of substrate #5. The following procedure was used: Material was aseptically cut into: 0.5×0.5 cm square sections, 0.2×2.0 cm strips, and 1.0×6.0 strips. All material was used in one-layer sections. Escherichia coli (ATCC 15597), and Staphylococcus aureus (ATCC 12600) were grown in five milliliters of tryptic soy broth (TSB) (Difco Laboratory, Detroit, Mich.) for 5 hours at 37° C. 0.5-mL of either bacterial culture was then added to molten (45° C.) sterile Tryptic Soy Agar (TSA) (Difco Laboratory, Detroit, Mich.). The mixture was then swirled and poured into a 15×100-mm petri dish. The gauze material was then aseptically placed onto the surface of the agar and the agar was allowed to solidify. The petri dish containing the sample was then incubated for 18 hours at 37° C. in 95% humidity. Zones of bacterial growth inhibition were then measured. Results are shown in Table E12.

TABLE E12
Results of zone of inhibition testing of Sample #32

Zone of inhibition
around sample (mm)

| Sample/section size | S. aureus | E. coli |
|---|---|---|
| SUB 5/1.5 × 1.5 | <0.1 | <0.1 |
| SUB 5/2.0 × 2.0 | <0.1 | <0.1 |
| SUB 5/1.0 × 5.0 | <0.1 | <0.1 |
| Sample #32/1.5 × 1.5 | <0.1 | <0.1 |
| Sample #32/2.0 × 2.0 | <0.1 | <0.1 |
| Sample #32/1.0 × 5.0 | <0.1 | <0.1 |

As shown in Table E12, no measurable zone of inhibition was observed around either the control or treated samples.

A study was conducted to determine the speed of antimicrobial action for the subject materials. Material similar in composition to sample #33 (code # 0712A) was used in this study, along with an untreated control (substrate #5). The following procedure was employed. Material was aseptically cut into approximately one inch square sections. 0712A gauze sample consisted of material in two layers, and SUB-5 samples were in 3 layers. Each sample section was individually placed in a sterile 100×15-mm petri dish and covered. Staphylococcus aureus (ATCC 12600) was grown in twenty-five milliliters of tryptic soy broth (TSB) (Difco Laboratory, Detroit, Mich.) for 6 hours at 37° C. The bacterial culture was then diluted in Fresh 1% TSB or 1×PBS containing 10% Fetal Bovine Serum (Sigma, St. Louis, Mo.) to a final concentration of approximately 10⁶-cfu/mL. One half (0.5) milliliter of the bacterial suspension was added to each gauze section. All gauze samples were inoculated in duplicates. The petri dish containing the inoculated sample was then incubated for the indicated time points at 37° C. in 95% humidity. Following incubation, the gauze material was aseptically placed into 50-mL conical centrifuge tubes. Twenty-five milliliters of sterile phosphate buffered saline (PBS) was then added to each tube. The tubes were shaken on a rotary shaker (Red Rotor PR70/75, Hoofer Scientific, CA) for 10 minutes. The eluant was then serially diluted. Tenfold dilutions were performed by the addition of 0.3-ML of sample to 2.7-mL of sterile PBS. Aliquots of each dilution or of the original undiluted sample were then aseptically spread plated in duplicates onto Tryptic Soy Agar (TSA) (Difco Laboratory, Detroit, Mich.) plates. The plates were incubated for 18 hours at 37° C. The colonies on the respective plates were counted and concentrations were determined. Results of this rate study are presented in Table E13.

TABLE E13
Effect of 0712A and Sub 5 gauze material on the inactivation of
Stapzhylococcus aureus at different exposure times.

Sample (and respective
bacterial count at
specified times)

| Time | Sub 5 | 0712A |
|---|---|---|
| 1 minute | 1.5 × 10⁵ | 3.0 × 10² |
| | 1.9 × 10⁵ | 3.1 × 10² |
| 10 minutes | 1.3 × 10⁵ | 2.0 × 10² |
| | 2.5 × 10⁵ | 8.0 × 10¹ |
| 20 minutes | 1.5 × 10⁵ | 8.0 × 10¹ |
| | 2.3 × 10⁵ | 1.2 × 10² |
| 30 minutes | 1.6 × 10⁵ | 1.1 × 10¹ |
| | 2.8 × 10⁵ | 2.1 × 10¹ |
| 60 minutes | 1.9 × 10⁵ | 1.2 × 10¹ |
| | 2.1 × 10⁵ | 1.9 × 10¹ |
| 4 hours | 3.3 × 10⁵ | 3 × 10⁰ |
| | 2.5 × 10⁵ | 1.2 × 10¹ |
| 8 hours | 4.0 × 10⁶ | <2.0 × 10⁰ |
| | 4.8 × 10⁶ | 4.0 × 10⁰ |
| 12 hours | 2.3 × 10⁷ | 6.0 × 10⁰ |

¹values represent cfu/mL of the 25-mL PBS solution used to elute the microorganisms from the gauze sections.

The data clearly indicates that significant antimicrobial activity is manifested very quickly. Approximately 99.8% of S. aureus is destroyed in as little as one minute.

Samples similar in composition to that of sample #31 in Table E3 were subjected to sterilization by several methods including: autoclaving, ethylene oxide exposure, gamma irradiation (2.5 Mrad), and electron beam irradiation (2.5 Mrad). No observable degradation of physical properties or loss of antimicrobial activity was observed.

Samples #43, #44 and #45 (see Table E3) were reacted for significantly shorter periods of time than the other samples listed; however, relatively high grafting yields were still obtained. This demonstrates that the process can be achieved quickly, which will have economic advantages for large-scale industrial application of this invention. It is likely that sufficiently high grafting yields can be obtained in 5 minutes or less under appropriate conditions.

Thus, the present invention teaches and demonstrates the effectiveness of a composition comprised of a substrate, preferably fibrous and water-insoluble, to which are attached by non-hydrolyzing covalent bonds a multitude of polymeric chains bearing quaternary ammonium groups. These chains predominantly contain more than one quaternary ammonium group per chain, and preferably have varying lengths and extend varying distances (measured at the molecular level) from the substrate. The present invention also teaches the manufacture of such compositions, where the preferred manufacture includes the steps of dewatering and drying the composition so it is available in a dry (not a hydrogel) form that is more capable of taking up wound exudate.

The present data demonstrates the superior effectiveness of compositions of the present invention compared with siloxane-based polymers such as taught by Blank et al. in U.S. Pat. No. 5,045,322. The '322 patent teaches attachment of monomeric siloxane-based quaternary compounds to super absorptive polymers. The siloxane-based compounds are sensitive to hydrolysis, as noted in the parent application. These siloxane compounds are expected to be more easily hydrolyzed than the acrylate polymers used in the present application. Furthermore, other polymers used in the present invention (such as those based on DADMAC or trialkyl(p-vinylbenzyl) ammonium chloride) are substantially more stable to hydrolysis than the bonds taught in the '322 patent.

In the case of siloxane-based antimicrobial agents, the chemical bonds which are susceptible towards hydrolysis are part of the backbone structure of the polymer. Hydrolysis of even a single siloxane linkage can result in the cleavage of several quaternary units (although the siloxane polymers in such systems are generally only a few units in length). In contrast, in the case of grafted acrylate polymers of the present invention, the grafted chains may be hundreds of units long. The ester linkages which attach the quaternary groups to the polymer backbone are inherently more stable than the linkages in the bulky siloxane quaternary units. Even so, it is possible that the acrylates can be hydrolyzed under extreme conditions. However, since the hydrolyzable group of the acrylate is not in the main chain of the polymer, this will not result in chain cleavage, so the loss under such unlikely, extreme conditions would be limited to a single quaternary unit per hydrolysis event.

Further, the antimicrobial effectiveness of a bulky molecule like the TMS siloxane used by Blank et al. is reduced somewhat by its steric hindrance. Since it can and does fold on itself, the number of such molecules that can be bonded to a given surface is limited as compared to smaller molecules. Further, the fact that the nitrogen atom can be blocked by other atoms in the molecule limits its positive charge density as well. The consequence of this is that the antimicrobial is less effective than one that can be attached to the same surface in greater numbers or density per unit area. Since the net positive charge on the nitrogen atom is related to the effectiveness of the antimicrobial, one that has more exposed positive atoms would theoretically be more effective. This can be shown by comparing the effectiveness of the Blank et al. compounds to any other quaternary compounds that have less steric hindrance. This is demonstrated in the results above, in Tables E4-E7. Another consequence is that in the presence of proteinaceous matter such as blood, urine, and tissue cells, the '322 compounds can be blocked more easily than quaternary polymers having a greater concentration of unhindered net positive charges. (See the parent application, PCT/US99/29091, and Table E7.)

A further shortcoming of the siloxane quaternary material disclosed according to Blank et al. is that it only provides a monolayer coverage of the surface. That is, the siloxane backbone molecules are not long-chain polymers. It is well known that siloxane chains more than a few units in length are particularly susceptible to hydrolysis, particularly those with bulky substituents such as the TMS monomer utilized in the '322 patent. This hydrolysis results in chain cleavage and loss of soluble antimicrobial. Such reactions occur as a result of cyclization or “back-biting” reactions (see: J. Semlyen, “Cyclic Polymers” Chapter 3, Elsevier, New York, 1986). By contrast, the surface according to the present invention is covered with polymeric chains composed of non-hydrolyzable carbon-carbon bonds, to which are bonded quaternary materials. Polymeric antimicrobials used according to the present invention are more effective than the monomeric antimicrobials described by Blank et al. (see Chen, Z. C., et al., “Quaternary Ammonium Functionalized Poly(propylene imine) Dendrimers as Effective Antimicrobials: Structure-Activity Studies”, Biomacromolecules 1, p 473-480 (2000); Ikeda, T., “Antibacterial Activity of Polycationic Biocides”, Chapter 42, page 743 in: High Performance Biomaterials, M. Szycher, ed., Technomic, Lancaster Pa., (1991); Donaruma, L. G., et al., “Anionic Polymeric Drugs”, John Wiley & Son, New York, (1978)). Thus, in order to obtain a high antimicrobial activity, a high surface area base material must be used with the siloxane quaternary materials. The Blank et al. patent describes placing this monolayer antimicrobial treatment onto powders, which are then used to make superabsorbent polymer gels. The powder has a very high surface area, and hence the gels contain a lot of antimicrobial. However, the Blank et al. gels have almost zero mechanical strength, (and must be contained inside some type of matrix in order to form a useable device). In contrast, the modified cellulose fibers of the present invention have inherent mechanical properties which allow them to be directly used as structural devices such as bandages.

A common understanding in the art is that an “enhanced surface area” would not apply to monolayer treatments such as the siloxane system described by Blank et al. That is, an enhanced surface area substrate is needed to achieve high quaternary content. According to the present invention, however, a high quaternary content may be achieved even on low surface area fibers such as cotton because the quaternary materials of the present invention are polymeric. An analogy may be made to the “fuzzy” structure of a pipe-cleaner to describe a single substrate fiber modified by the currently-described method—that is, each “hair” of the pipe cleaner represents a polymer chain which has an antimicrobial group on substantially each monomer that makes up the polymer. The present applicants have actually attempted use of a Dow Corning product (TMS—the same compound described by Blank et al.) to treat fabrics, and have found that a significantly lower amount of quaternary antimicrobial groups could be applied. The bactericidal activity of the TMS treated fabrics was several orders of magnitude lower than the fabrics treated with polymeric quaternary materials of this invention. The inventors further found that the TMS-treated samples became water-repellent. This effect was reported by Blank et al. (see U.S. Pat. No. 5,035,892; column 12, line 57). This impairment of absorbency is undesirable in a product intended for use as an absorbent. Furthermore, the siloxane monomer has a higher MW than the monomers of the present invention. As a result, the effective quaternary material content (number of positively-charged sites per gram of material) is further reduced as compared to that of the present invention. Finally, the present application further discloses use of neutral or negatively charged antimicrobial polymers, which is neither disclosed nor suggested according to Blank et al.

It should also be noted that the mechanism of action of quaternary compounds is directed towards the cell membrane of the target organism. This process has been described as a mechanical “stabbing” (on a molecular level) which causes rupture of the cell membrane. Thus, it is not possible for pathogenic organisms to develop resistance as observed for most antibiotics.

The following examples demonstrate the use of the various initiators described above for the formation of graft copolymers between cellulose and quaternary-containing vinyl monomers:

### Example 15

### Grafting of Quaternary Ammonium Polymers onto Cellulose Fabric

A solution of 0.4 gram SPS, 65 mL distilled water, and 20 mL of Ageflex FM1Q75MC ([2-(methacroyloxy)ethyl]trimethylammonium chloride, 75 wt % solution in water, Ciba Specialty Chemicals Corporation) was placed into a 250 mL screw-top glass jar, and then sparged with argon gas to remove dissolved oxygen. One sheet of rayon non-woven gauze fabric (Sof-Wick, manufactured by J&J) was dried, weighed (2.00 grams total), and placed into the above solution. The jar was flushed with nitrogen, capped, and placed into a 60° C. oven overnight. The fabric sample was then removed, thoroughly washed with tap water, and then dried. The final weight of the samples was 2.49. This represents a grafting yield of 19.4%. The sample was bright white in color, and showed no degradation or discoloration. Testing with a 1% solution of fluorescein dye, followed by thorough rinsing left a bright orange color which indicates the presence of quaternary ammonium groups grafted to the fabric surface. The sample was aseptically cut into approximately one inch² square sections. Each sample section was placed in a sterile 100×15-mm petri dish and covered. Escherichia coli (ATCC 15597) were grown in twenty five milliliters of tryptic soy broth (TSB) (Difco Laboratory, Detroit, Mich.) for 16 hours at 37° C. Each bacterial culture was then diluted a hundred-fold in Fresh phosphate buffered saline (PBS) containing 10% Fetal Bovine Serum (FBS, Sigma, St. Louis, Mo.) to a final concentration of 7.2×10⁷-cfu/mL coli. One-half milliliter of the bacterial suspension was added to each material section. All samples were inoculated in triplicates. The petri dish containing the inoculated sample was then covered and incubated for 18 hours at 36° C. in 100% humidity. Following incubation, the gauze material was aseptically placed into 50-mL conical centrifuge tubes. Twenty-five milliliters of sterile PBS was then added to each tube. The tubes were gently shaken on a rotary shaker (Red Rotor PR70/75, Hoofer Scientific, CA) for 30 minutes. The eluant of samples were then serially diluted thousand and ten thousand-fold by the addition of 1.0 or 0.1-mL of sample to 100-mL of sterile PBS. 0.1-mL aliquots of the diluted samples were then aseptically spread plated onto Tryptic Soy Agar (TSA) (Difco Laboratory, Detroit, Mich.) plates. Additionally, 0.1-mL and 0.33-mL aliquots of the undiluted PBS samples containing the gauze were also aseptically spread plated onto TSA. The plates were incubated for 18 hours at 37° C. The colonies on the respective plates were counted and concentrations were determined. It was found that a greater than 6-log reduction of bacteria was obtained (versus untreated rayon gauze control).

### Example 16

### Preparation of Quaternary-Grafted Rayon Samples

The method of Example 15 was used to prepare quaternary-grafted rayon samples. In this experiment, samples were not heated, but instead left at room temperature (25° C.) for various lengths of time. The following results (% grafting vs. reaction time) were obtained: (2 hours—5.5%; 4 hours—13.4%; 69 hours—17.4%).

### Example 17

### Preparation of Quaternary-Grafted Rayon Samples

The method of Example 15 was used to prepared quaternary-grafted rayon samples. In this experiment, samples were heated for shorter lengths of time before being removed from the oven and washed. The following results (% grafting vs. reaction time) were obtained: (30 minutes—9.5%; 60 minutes—14.4%; 4 hours—15.4%).

### Example 18

### Preparation of Quaternary-Grafted Cotton Samples

The method of Example 15 was used, except the rayon gauze substrate was replaced with bulk cotton (7.08 grams). The following solution was used: 1.5 grams SPS, 210 mL distilled water, and 45 mL Ageflex FM1Q75MC. The sample was heated at 60° C. overnight. The grafting yield was 4.8%. The sample was tested against E. coli bacteria as described in Example 15. A greater than 6-log reduction of viable bacteria was observed.

### Example 19

### Preparation of Quaternary-Grafted Samples

The method of Example 16 was repeated using a 2 hour reaction time at 60° C. In this experiment the step of sparging with argon gas was omitted. The grafting yield was 10.3%.

### Example 20

### Preparation of Quaternary-Grafted Woven Cotton Samples

The method of Example 15 was repeated except that 5.05 grams of woven cotton bedsheet material was used as a substrate (1 gram SPS, 70 mL distilled water, and 30 mL Ageflex FM1Q75MC). The grafting yield was found to be 2.8%. The grafted material was tested against E. coli bacteria as described in Example 15. A greater than 6-log reduction of viable bacteria was observed.

### Example 21

### Preparation of Quaternary-Grafted Samples

The method of Example #3 was repeated except that a solution of 3% aqueous hydrogen peroxide (5 mL) was used in place of SPS. The grafting yield was found to be 15.8%.

### Example 22

### Preparation of Quaternary-Grafted Samples

The method of Example 15 was repeated except that 0.50 gram VA-057 (2,2′-Azobis[N-(2-carboxyethyl)-2-methylpropionamidine]tetrahydrate, available from Wako Specialty Chemicals) was used in place of SPS. A 9.5% grafting yield was obtained.

### Example 23

### Preparation of Quaternary-Grafted Samples

The method of Example 15 was repeated, except that a solution of 3% aqueous hydrogen peroxide (3 mL) was used in addition to SPS. A 24.5% grafting yield was obtained.

### Example 24

### Preparation of a Superabsorbent Polymer Network

This example illustrates the preparation of a superabsorbent polymer network (SAP). The method of Example 15 was used, except that 0.5 gram of difunctional acrylate crosslinking agent (SR344, polyethylene glycol diacrylate, Sartomer Chemical Co.) was also used. The sample was heated for 2 hours at 60° C., and a solid gel was formed. Excess gel was scraped away from the substrate which was then washed by soaking it in water for greater than 24 hours. The sample was then dried in air. The resulting white fabric sheet was found to be capable of absorbing 25 times its own weight of water.

### Example 25

### Preparation of DADMAC-Treated Mica Powder

Two grams of mica particles (<38 μm particle size) were placed into a solution of 0.1 g AMBP, 10 g of 65% DADMAC, and 10 g of water, then sparged with argon gas. The mixture was sealed in a jar under argon atmosphere and heated for 90 minutes at 80° C. The mixture was suspended in water (4 L), allowed to settle for several hours, then resuspended in fresh water. After settling overnight, the mica powder wash washed several times in distilled water (50 mL aliquots), and washed by repeated shaking and centrifugation. The powder was then dried in a vacuum oven. Testing of the treated mica with a 1% solution of bromthymol blue dye produced a dark blue coloration after washing. Untreated mica powder tested in a similar manner showed no dye absorption. The powder was tested for antimicrobial activity against E. coli according to the method in the above example. Antimicrobial activity was high, (six log reduction) and no viable bacteria were observed.

The following examples provide detailed written description and enablement for various aspects of the present invention whereby polyionic substrates are created and charged with ionic biologically or chemically active compounds for adherence and sustained release:

### Example 26

NIMBUS-10™, (a nonwoven rayon gauze material graft polymerized with diallyldimethylammonium chloride (DADAMC), and containing approximately 10 weight % poly(DADMAC)), and SofWick, a commercially available rayon gauze material manufactured by Johnson & Johnson were used as substrates. The NIMBUS™ material was prepared via modification of the SofWick substrate. Each substrate measured approximately 40 square inches. Substrates were dried at 60° C. for 30 minutes and then weighed. Both samples were trimmed to weigh exactly 1.00 grams each. A 0.5 weight % solution of Cefazolin Sodium USP (Geneva Pharmaceuticals) was prepared. Each sample was placed in a 50 mL screw-cap polypropylene centrifuge tube, along with 30 mL of the Cefazolin solution and the tubes were placed on a rotating agitator for 3 hours. The samples were removed from the solutions, then squeezed to remove excess solution, dried at 60° C. for 2 hours, then weighed. The NIMBUS™ sample weighed 1.05 grams, whereas the SofWick sample weighed 1.00 grams. The gravimetric analysis indicated that substantially more drug was absorbed by the NIMBUS™ sample, compared to the untreated rayon substrate. The extraction liquid was saved for analysis (Solution E1—NIMBUS™; Solution F1—SofWick).

The dried samples were placed in separate 50 mL centrifuge tubes containing 25 mL of distilled water, then placed in the rotating agitator overnight at room temperature. The samples were then removed, squeezed to remove excess solution, dried at 60° C. for 2 hours, then weighed. The NIMBUS™ sample weighed 1.03 grams, and the SofWick sample weighed 0.98 grams. Gravimetric analysis indicated that only a portion of the bonded drug had been released from the NIMBUS™ sample. The extraction liquid was saved for analysis (Solution E2-NIMBUS™; Solution F2—SofWick). This extraction procedure was repeated four additional times to yield two series of six extraction liquids (NIMBUS™: E1-E6; and SofWick F1-F6).

The extract solutions were tested for antimicrobial activity by placing single 20 microliter drops of the solutions at marked locations on an agar culture plate spread with ˜3×10³ CFU (continuous lawn) of E. coli bacteria. Plates were incubated overnight at 37° C., and the diameter of the “zone of inhibition”, or “ZOI” was measured. The size of this zone corresponds to the antibacterial activity of the extract solutions. Results are listed below in Table E14.

Solutions E-1 and F-1 were discarded without analysis, since they were likely to contain non-bonded drug. Solution F-3 showed no inhibition of bacterial growth, indicating that all of the drug had been removed in 3 or less washings, thus further samples in this series were not analyzed.

TABLE E14

| Sample | ZOI diameter (mm) |
|---|---|
| Cefazolin 1% | 40 |
| Cefazolin 0.1% | 18 |
| Cefazolin 0.01% | 0 |
| E-2 (NIMBUS ™) | 23 |
| E-3 | 22 |
| E-4 | 23 |
| E-5 | 18 |
| E-6 | 16 |
| F-2 (SofWick) | 21 |
| F-3 | 0 |

The superior binding, and controlled-release properties of NIMBUS™ for the Cefazolin drug versus untreated SofWick are clearly demonstrated.

### Example 27

This example is similar to Example 26 (above), except that Penicillin G Potassium (PG) (Squibb-Marsam) was used as the drug, and antibacterial efficacy was tested against Staph. aureus instead of E. coli. Samples (NIMBUS™: Sample “G”, and unmodified SofWick: Sample “H”) were soaked in 25 mL of 5% PG solution for 2 hours, then squeezed to remove excess solution, dried and weighed. Samples were then washed with 25 mL of distilled water for one hour, then dried and weighed. These wash solutions (G-1 and H-1) were saved for analysis. The samples were then subjected to two additional washings with 25 mL distilled water, without drying in between washings (G-2, G-3, and H-2, H-3). Samples were then dried and weighed. Extract solutions were tested for antimicrobial activity, and the results are shown below. Extract H-3 was found to have zero antimicrobial activity, and thus further extractions were not performed on sample H. Sample G was then subjected to ten additional extractions with 25 mL of distilled water, and only dried and weighed between extractions G8 and G9, and after G13. All extracts were tested for antimicrobial activity, and results are reported below in Tables E15 and E16.

TABLE E15

| | Sample H (SofWick) | Sample G (NIMBUS ™) |
|---|---|---|
| Initial sample weight | 0.971 g | 1.053 g |
| After drug loading | 1.058 g | 1.269 g |
| After washing 1x | 0.979 g | 1.177 g |
| After washing 3x | 0.973 g | 1.147 g |
| After washing 8x | n.d. | 1.120 g |
| After washing 13x | n.d. | 1.090 g |

TABLE E16

| Sample | ZOI diameter (mm) |
|---|---|
| Penicillin G (1.0%) | 56 |
| Penicillin G (0.1%) | 44 |
| Penicillin G (0.01%) | 0 |
| G-1 (NIMBUS ™) | 56 |
| G-2 | 48 |
| G-3 | 46 |
| G-4 | 46 |
| G-5 | 46 |
| G-6 | 46 |
| G-7 | 46 |
| G-8 | 46 |
| G-9 | 46 |
| G-10 | 46 |
| G-11 | 46 |
| G-12 | 46 |
| G-13 | 46 |
| H-1 (untreated SofWick) | 56 |
| H-2 | 48 |
| H-3 | 0 |

The sample clearly absorbs more penicillin than the untreated rayon substrate, and binds and releases aliquots of the drug, even after thirteen extractions with distilled water.

### Example 28

A repeat of the method of Example 27 was used, except that the extraction solvent used was phosphate buffered saline (“PBS”, pH=7.4, Fisher Scientific), and samples were not dried and weighed between extractions. Samples of NIMBUS™ (Sample I, initial weight=1.024 g) and SofWick (Sample J, initial weight=1.011 g) were each soaked in 20 Ml of ˜4% penicillin G solution overnight, and excess solution was removed by squeezing. Samples were then washed with 25 mL distilled water for one hour with agitation, squeezed to remove excess liquid, and then subjected to five sequential extractions using 25 mL of PBS for one hour at room temperature. Samples were squeezed to remove excess solution between extractions. Extracts were tested for antibacterial activity against S. aureus according to the procedure outlined above. The results are summarized below in Table E17.

TABLE E17

| | NIMBUS ™: ZOI | SofWick: ZOI |
|---|---|---|
| # of extractions | diameter (mm) | diameter (mm) |
| 1 | 55 | 45 |
| 2 | 50 | 34 |
| 3 | 40 | 11 |
| 4 | 30 | 0 |
| 5 | 10 | 0 |

Again, the results clearly show higher initial drug concentration, and prolonged release from the NIMBUS™ material. Use of saline as the extractant accelerated release of the drug from the substrate; however, the binding effect is still readily apparent.

### Example 29

This example demonstrates the stabilization of pyrithione by a cationic cellulose surface. The method of Example 27 was used, except that sodium pyrithione (“SP”, Acros Chemical) was used instead of penicillin. One gram samples of NIMBUS™ (sample K), and untreated SofWick (sample L) were each soaked overnight in 25 ml of 0.5% SP solution. Samples were removed and squeezed to remove excess liquid. Samples were then washed in 25 mL of distilled water for one hour with agitation, then squeezed to remove excess liquid. Each sample was subjected to four sequential extractions using 25 mL of distilled water for one hour at room temperature with agitation. Samples were squeezed to remove excess solution between extraction cycles. The extracts were tested for antibacterial activity against S. aureus using the procedure described above. Results are shown below in Table E18:

TABLE E18

| | NIMBUS ™: ZOI | SofWick: ZOI |
|---|---|---|
| # of extractions | Diameter (mm) | Diameter (mm) |
| 1 | 12 | 0 |
| 2 | 11 | 0 |
| 3 | 14 | 0 |
| 4 | 12 | 0 |

SP control solutions exhibited the following ZIO (0.1% SP: 26 mm; 0.01% SP: 12 mm). This example clearly shows the binding and stabilization of SP by the cationic cellulose substrate (NIMBUS™).

Having generally described this invention, including the best mode thereof, those skilled in the art will appreciate that the present invention contemplates the embodiments of this invention as defined in the following claims, and equivalents thereof. However, those skilled in the art will appreciate that the scope of this invention should be measured by the claims appended hereto, and not merely by the specific embodiments exemplified herein. Those skilled in the art will also appreciate that more sophisticated technological advances will likely appear subsequent to the filing of this document with the Patent Office. To the extent that these later developed improvements embody the operative principles at the heart of the present disclosure, those improvements are likewise considered to come within the ambit of the following claims.

1. A device for the treatment of wounds comprising:
a) an absorbent wound dressing material having incorporated therein inherent non-leachable antimicrobial activity and inherent non-leachable anti-protease activity; and
b) a releasable antimicrobial agent and a releasable anti-protease agent that are ionically stabilized within the device so as to be released from the device in a controlled manner.

2. The device of claim 1, further comprising one or more releasable bioactive agents which aid in wound healing, selected from the group consisting of growth factors, vitamins, antioxidants, anti-inflammatories, antimicrobials, anti-proteases, steroids and nutrients.

3. The device of claim 1, wherein the releasable antimicrobial agent and the releasable anti-protease agent are one and the same substance.

4. The device of claim 3, wherein the releasable antimicrobial agent and the releasable anti-protease agent are doxycycline.

5. The device of claim 1, wherein one or both of the inherent non-leachable antimicrobial activity and the inherent non-leachable antiprotease activity is provided by polymeric quaternary ammonium molecules.

6. The device of claim 5, wherein said polymeric quaternary ammonium molecules comprise poly(dimethyldiallylammonium chloride) also known as polyDADMAC; quaternary ammonium derivatives of poly(acrylic) acid or of poly(methacrylic) acid; poly(vinylbenzyl)trimethylammonium chloride; or a polyquaternium.

7. The device of claim 5, wherein the polymeric quaternary ammonium molecules are non-leachably bonded to the wound dressing material via covalent chemical bonds.

8. The device of claim 5, wherein the polymeric quaternary ammonium molecules are non-leachably bonded to the wound dressing material via ionic bonds.

9. The device of claim 5, wherein said polymeric quaternary ammonium molecules have a crosslinked network structure that forms a hydrogel in water.

10. The device of claim 5, wherein said polymeric quaternary ammonium molecules have a crosslinked network structure formed via chemical crosslinking of said polymeric quaternary ammonium molecules.

11. The device of claim 9, wherein said polymeric quaternary ammonium molecules have crosslinked network structure which comprises a polyelectrolyte complex formed between said polymeric quaternary ammonium molecules and polymeric anionic molecules.

12. The device of claim 11, wherein said polymeric anionic molecules comprise one or more of carboxymethyl cellulose, also known as CMC, alginate, or polyacrylate.

13. The device of claim 1, wherein said wound device comprises one or more of cellulose, a cellulose-based material, a polysaccharide, a textile fabric, gauze, fibers, a synthetic polymer, a superabsorbent material, or a protein.

14. The device of claim 13, wherein the cellulose-based material is one or more of cotton, rayon, CMC, hydroxyethyl cellulose, paper, or woodpulp, and wherein the polysaccharide is one or more of dextran, chitosan, alginate, or starch, and wherein the protein is collagen.

15. The device of claim 1, wherein said inherent non-leachable anti-protease activity is provided by a polymer with a multiplicity of anionic sites.

16. The device of claim 15, where said polymer with a multiplicity of anionic sites is one or more of CMC, alginate, collagen or polyacrylate.

17. The device of claim 1, wherein said releasable antimicrobial agent comprises an antibiotic or polymeric quaternary ammonium molecule.

18. The device of claim 17, wherein said releasable antimicrobial agent is one or more of tetracycline, doxycycline and poly(DADMAC).

19. The device of claim 1, wherein said releasable antiprotease agent is one or more of ilomastat, doxycycline, minocycline, collagen, CMC or poly(DADMAC).

20. The device of claim 1, wherein said inherent non-leachable antimicrobial agent and said inherent non-leachable anti-protease agent is one and the same.

21. The device of claim 1, wherein said releasable bioactive agent is one or more growth factors, vitamins, nutritive factors, and anti-inflammatories.

22. The device of claim 21, wherein the growth factor is epidermal growth factor (EGF), platelet derived growth factor (PDGF), or vascular endothelial growth factor (VEGF).

23. An absorbent antimicrobial material comprising a substantially carboxymethylated cellulosic substrate and poly(DADMAC) non-leachably attached to said substrate, wherein a sufficient amount of said poly(DADMAC) is attached to said substrate to form a polyelectrolyte network, and wherein said polyelectrolyte network permits the degree of swelling of said material to range from about 10 times up to about 20 times of the dry material, and wherein said polyelectrolyte network diminishes the dissolution of the substantially carboxymethylated cellulosic substrate upon exposure to aqueous fluids, and wherein said polyelectrolyte network permits the incorporation and release of a bioactive agent in a controlled manner.

24. A method for the preparation of a non-leaching antimicrobial-coated device, comprising the steps of:
a. immersing all or a portion of a substrate into a solution comprising a sufficient quantity of monomer bearing at least one antimicrobial group per monomer molecule, and a sufficient quantity of catalyst to sustain polymerization reactions to sufficiently coat said substrate to impart an antimicrobial characteristic;
b. maintaining the contact of said substrate with said solution under acceptable conditions for a sufficient period of time to complete said reaction, wherein said reactions comprise forming polymers of varying lengths, and forming covalent, non-siloxane bonds between the majority of said polymers of varying lengths and binding sites on said substrate;
c. rinsing said substrate sufficiently to remove non-polymerized monomer molecules, non-stabilized polymeric molecules, and catalyst;
d. drying said substrate to a desired low moisture content, such that the substrate is not a hydrogel; and
e. contacting the thus prepared substrate with sufficient anionic or cationic biologically or chemically active compound to achieve ionic association between said compound and said substrate.

25. The method of claim 24, additionally comprising the step of maintaining the solution and gases in contact with the solution free of oxygen by sparging with an inert gas.

26. The method of claim 24, wherein said rinsing is with an aqueous solution, and additionally comprising the step of dewatering the substrate after the rinsing step.

27. The method of claim 24, wherein the catalysts is selected from the group consisting of a cerium salt, a peroxide, a persulfate, and, an Azo catalyst, and a photolabile or thermolabile catalyst.

28. A material comprising a substrate and an enhanced surface area, said enhanced surface area comprising a multitude of non-hydrolyzable, non-leachable polymeric molecules covalently bonded by non-siloxane bonds to said substrate; wherein said non-hydrolyzable, non-leachable polymeric molecules comprise a multitude of antimicrobial groups attached to said non-hydrolyzable, non-leachable polymeric molecules by covalent bonds; and wherein a sufficient number of said non-hydrolyzable, non-leachable polymeric molecules are covalently bonded to sites of said substrate to render the material antimicrobial, or receptive to avid binding of negatively charged dye molecules, when exposed to aqueous fluids, menses, bodily fluids, skin, cosmetic compositions, or wound exudates, wherein said material has associated therewith a plurality of anionically charged biologically or chemically active compounds.

29. The material of claim 28, wherein said antimicrobial groups comprise at least one quaternary ammonium structure.

30. The material of claim 28, wherein said antimicrobial groups comprise at least one non-ionic structure.

31. The material of claim 30, wherein said at least one non-ionic structure comprises a biguanide.

32. The material of claim 28, wherein said non-hydrolyzable, non-leaching polymeric molecules have an average degree of polymerization selected from about 5 to 1000, 10 to 500, and 10 to 100.

33. The material of claim 28, wherein said material comprises all or part of a wound dressing, sanitary pad, a tampon, an intrinsically antimicrobial absorbent dressing, a diaper, toilet paper, a sponge, a sanitary wipe, isolation and surgical gowns, gloves, surgical scrubs, sutures, sterile packaging, floor mats, lamp handle covers, burn dressings, gauze rolls, blood transfer tubing or storage container, mattress cover, bedding, sheet, towel, underwear, socks, cotton swabs, applicators, exam table covers, head covers, cast liners, splint, paddings, lab coats, air filters for autos, planes or HVAC systems, military protective garments, face masks, devices for protection against biohazards and biological warfare agents, lumber, meat or fish packaging material, apparel for food handling, paper currency, powder, and other surfaces required to exhibit a non-leaching antimicrobial property and to release over time portions of said biologically or chemically active compound.

34. The material of claim 28, wherein said substrate is comprised, in whole or in part, of cellulose, or other naturally-derived polymers.

35. The material of claim 28, wherein said substrate is comprised, in whole or in part, of synthetic polymers including, but not limited to: polyethylene, polypropylene, nylon, polyester, polyurethane, or silicone.

36. The material of claim 28, wherein said attachment of said non-hydrolyzable, non-leachable polymer to said substrate is via a carbon-oxygen-carbon bond, also known as an ether linkage, a carbon-carbon bond, and mixtures thereof.

37. The material of claim 36, wherein a cerium-containing catalyst, a peroxide containing catalyst, an Azo catalyst, a redox initiator, a thermolabile or photolabile catalyst catalyzes formation of said ether linkage or said carbon-carbon bond.

38. The material of claim 28, wherein said non-hydrolyzable, non-leachable polymeric molecules are formed by polymerization of allyl- or vinyl-containing monomers.

39. The material of claim 38, wherein said allyl- or vinyl-monomers are selected from the group consisting of: styrene derivatives, allyl amines, and ammonium salts.

40. The material of claim 38, wherein said allyl- or vinyl-monomers are selected from the group consisting of: acrylates, methacrylates, acrylamides, and methacrylamides.

41. The material of claim 40, wherein said allyl- or vinyl-containing monomers are selected from the group consisting of: compounds of the structure CH₂═CR—(C═O)—X—(CH2)ₙ—N⁺R′R″R′″//Y⁻; wherein, R is hydrogen or methyl, n equals 2 or 3, X is either O, S, or NH, R′, R″, and R′″ are independently selected from the group consisting of H, C1 to C16 alkyl, aryl, arylamine, alkaryl, and aralkyl, and Y− is an acceptable anionic counterion to the positive charge of the quaternary nitrogen; diallyldimethylammonium salts; vinyl pyridine and salts thereof; and vinylbenzyltrimethylammonium salts.

42. The material of claim 41, where said allyl- or vinyl-containing monomers are selected from the group consisting of: dimethylaminoethyl methacrylate:methyl chloride quaternary; and dimethylaminoethyl methacrylate:benzyl chloride quaternary.

43. The material of claim 33, wherein said powder is mica.

44. A superabsorbent material for absorbing biological fluids, comprising a substrate and an enhanced surface area, said enhanced surface area comprising a multitude of non-hydrolyzable, non-leachable polymeric molecules covalently bonded by non-siloxane bonds to said substrate; wherein said non-hydrolyzable, non-leachable polymeric molecules comprise a multitude of antimicrobial groups attached to said non-hydrolyzable, non-leachable polymeric molecules by covalent bonds; and wherein a sufficient number of said non-hydrolyzable, non-leachable polymeric molecules are covalently bonded to sites of said flexible substrate to render the material antimicrobial when exposed to aqueous fluids, menses, bodily fluids, or wound exudates; wherein said superabsorbent material is capable of absorbing about 30 or more times its own weight of water or other fluids in a single instance; and wherein said absorbing capacity is the result of branching or crosslinking of said non-hydrolyzable, non-leachable polymeric molecules, wherein said material has associated therewith a plurality of anionically charged biologically or chemically active compounds.

45. The material of claim 44, wherein said antimicrobial groups comprise at least one quaternary ammonium structure.

46. The material of claim 44, wherein said antimicrobial groups comprise at least one non-ionic structure.

47. The material of claim 46, wherein said at least one non-ionic structure comprises a biguanide.

48. The material of claim 44, wherein said material comprises all or part of a wound dressing, sanitary pad, a tampon, an intrinsically antimicrobial absorbent dressing, a diaper, toilet paper, a sponge, a sanitary wipe, food preparation surfaces, gowns, gloves, surgical scrubs, sutures, needles, sterile packings, floor mats, lamp handle covers, burn dressings, gauze rolls, blood transfer tubing or storage container, mattress cover, bedding, sheet, towel, underwear, socks, cotton swabs, applicators, exam table covers, head covers, cast liners, splint, paddings, lab coats, air filters for autos planes or HVAC systems, military protective garments, face masks, devices for protection against biohazards and biological warfare agents, lumber, meat packaging material, paper currency, powders, and other surfaces required to exhibit a non-leaching antimicrobial or enhanced dye binding properties, and to release over time portions of said biologically or chemically active compound.

49. The material of claim 44, wherein said substrate is comprised, in whole or in part, of cellulose, or other naturally-derived polymers.

50. The material of claim 44, wherein said substrate is comprised, in whole or in part, of synthetic polymers including, but not limited to: polyethylene, polypropylene, nylon, polyester, polyurethane, or silicone.

51. The material of claim 44, wherein said attachment of said non-hydrolyzable, non-leachable polymer to said substrate is via a carbon-oxygen-carbon bond, also known as an ether linkage, a carbon-carbon bond, or mixtures thereof.

52. The material of claim 51, wherein a cerium-containing catalyst, a peroxide containing catalyst, an Azo catalyst, a thermolabile or photolabile catalyst catalyzes formation of said ether linkage or said carbon-carbon linkage, or mixtures thereof.

53. The material of claim 44, wherein said non-hydrolyzable, non-leachable polymeric molecules are formed by polymerization of allyl- or vinyl-containing monomers.

54. The material of claim 53, wherein said allyl- or vinyl-monomers are selected from the group consisting of: styrene derivatives; and allyl amines or ammonium salts.

55. The material of claim 53, wherein said allyl- or vinyl-monomers are selected from the group consisting of: acrylates, methacrylates, acrylamides, and methacrylamides.

56. The material of claim 55, wherein said allyl- or vinyl-containing monomers are selected from the group consisting of: compounds of the structure CH₂═CR—(C═O)—X—(CH2)ₙ—N⁺R′R″R′″/Y⁻; wherein, R is hydrogen or methyl, n equals 2 or 3, X is either O, S, or NH, R′, R″, and R′″ are independently selected from the group consisting of H, C1 to C16 alkyl, aryl, arylamine, alkaryl, and aralkyl, and Y− is an acceptable anionic counterion to the positive charge of the quaternary nitrogen; diallyldimethylammonium salts; vinyl pyridine and salts thereof; and vinylbenzyltrimethylammonium salts.

57. The material of claim 56, where said allyl- or vinyl-containing monomers are selected from the group consisting of: dimethylaminoethyl methacrylate:methyl chloride quaternary; and dimethylaminoethyl methacrylate:benzyl chloride quaternary.

58. An inherently antimicrobial composition comprising:
a. a substrate;
b. a coating, layer, or enhanced surface area on said substrate, comprised of a plurality of polymeric molecules of variable lengths bearing antimicrobial groups, wherein said polymeric molecules are covalently, non-leachably bound to said substrate, and wherein said coating, layer, or enhanced surface area exhibits antimicrobial activity due to the presence of said antimicrobial groups; and
c. ionically associated biologically or chemically active compounds which are released from said substrate and coating layer over a period of time.

59. The composition of claim 58, wherein said antimicrobial groups comprise at least one quaternary ammonium structure.

60. The composition of claim 58, wherein said antimicrobial groups comprise at least one non-ionic structure.

61. The composition of claim 60, wherein said at least one non-ionic structure comprises a biguanide.

62. The composition of claim 58, wherein said material comprises all or part of a wound dressing, sanitary pad, a tampon, an intrinsically antimicrobial absorbent dressing, a diaper, toilet paper, a sponge, a sanitary wipe, food preparation surfaces, gowns, gloves, surgical scrubs, sutures, needles, sterile packings, floor mats, lamp handle covers, burn dressings, gauze rolls, blood transfer tubing or storage container, mattress cover, bedding, sheet, towel, underwear, socks, cotton swabs, applicators, exam table coves, head covers, cast liners, splint, paddings, lab coats, air filters for autos, planes or HVAC systems, military protective garments, face masks, devices for protection against biohazards and biological warfare agents, lumber, meat packaging material, paper currency, powders, and other surfaces required to exhibit a non-leaching antimicrobial or enhanced dye binding properties, and to release over time portions of said biologically or chemically active compound.

63. The composition of claim 58, wherein said substrate is comprised, in whole or in part, of cellulose, or other naturally-derived polymers.

64. The composition of claim 58, wherein said substrate is comprised, in whole or in part, of synthetic polymers including, but not limited to: polyethylene, polypropylene, nylon, polyester, polyurethane, or silicone.

65. The composition of claim 58, wherein said attachment of said non-hydrolyzable, non-leachable polymeric molecule to said substrate is via a carbon-oxygen-carbon bond, also known as an ether linkage, via a carbon-carbon bond, or mixtures thereof.

66. The composition of claim 65, wherein a cerium-containing catalyst, a peroxide containing catalyst, an Azo catalyst, a thermolabile or photolabile catalyst catalyzes formation of said ether linkage or said carbon-carbon linkage, or mixtures thereof.

67. The composition of claim 58, wherein said non-hydrolyzable, non-leachable polymeric molecules are formed by polymerization of allyl- or vinyl-containing monomers.

68. The composition of claim 67, wherein said allyl- or vinyl-monomers are selected from a group consisting of: styrene derivatives; allyl amines and ammonium salts.

69. The composition of claim 67, wherein said allyl- or vinyl-monomers are selected from the group consisting of: acrylates, methacrylates, acrylamides, and methacrylamides.

70. The composition of claim 69, wherein said allyl- or vinyl-containing monomers are selected from the group consisting of: compounds of the structure CH₂═CR—(C═O)—X—(CH2)ₙ—N⁺R′R″R′″//Y⁻; wherein, R is hydrogen or methyl, n equals 2 or 3, X is either O, S, or NH, R′, R″, and R′″ are independently selected from the group consisting of H, C1 to C16 alkyl, aryl, arylamine, alkaryl, and aralkyl, and Y− is an acceptable anionic counterion to the positive charge of the quaternary nitrogen; diallyldimethylammonium salts; vinyl pyridine and salts thereof; and vinylbenzyltrimethylammonium salts.

71. The composition of claim 70, where said allyl- or vinyl-containing monomers are selected from the group consisting of: dimethylaminoethyl methacrylate:methyl chloride quaternary; and dimethylaminoethyl methacrylate:benzyl chloride quaternary.

72. The antimicrobial composition of claim 71, wherein said substrate is selected from the group consisting of: woven or nonwoven flexible matrices, wherein said composition is formed into the shape of a wound dressing and a powder.

73. The antimicrobial composition of claim 71, wherein said coating absorbs aqueous liquids.

74. The antimicrobial composition of claim 71, wherein said substrate is wood, lumber, or an extract or a derivative of wood fiber.

75. An antimicrobial-coated composition for destruction of microbes contacting said composition, comprising:
a. a substrate onto which a coating of antimicrobial polymers is bonded;
b. said coating, formed of an effective amount of polymeric molecules having a multiplicity of quaternary ammonium groups, wherein said polymeric molecules are non-leachably and covalently bonded to surface sites of said substrate, wherein said polymers do not form using siloxane bonds, and wherein said coating is absorbent of aqueous liquids, and
c. associated anionic biologically active or chemically active compound; whereby said multiplicity of quaternary ammonium groups act to destroy microbes coming in contact with said groups as well as to bind and release said anionic biologically active or chemically active compound.

76. A composition comprising mica which has been subjected to coating, grafting, binding or adhesion of a quaternary amine polymer, followed by association of anionic biologically or chemically active compounds with said quaternary amine polymer.

77. The method according to claim 24 wherein said sufficient anionic or cationic biologically or chemically active compound to achieve ionic association between said compound and said substrate are selected from the group consisting of: antibiotics, analgesics, anti-inflammatories, strong oxidizing agents, matrix metalloproteinase inhibitors, proteins, peptides, fragrances, and antifungal.

78. The composition according to claim 28, wherein said plurality of anionically charged biologically or chemically active compounds are selected from the group consisting of: antibiotics, analgesics, anti-inflammatories, strong oxidizing agents, matrix metalloproteinase inhibitors, proteins, peptides, fragrances, and antifungals.

79. The composition according to claim 44, wherein said plurality of anionically charged biologically or chemically active compounds are selected from the group consisting of: antibiotics, analgesics, anti-inflammatories, strong oxidizing agents, matrix metalloproteinase inhibitors, proteins, peptides, fragrances, and antifungals.

80. The composition according to claim 58, wherein said ionically associated biologically or chemically active compounds which are released from said substrate and coating layer over a period of time are selected from the group consisting of: antibiotics, analgesics, anti-inflammatories, strong oxidizing agents, matrix metalloproteinase inhibitors, proteins, peptides, fragrances, and antifungals.

81. The composition according to claim 75 wherein said associated anionic biologically active or chemically active compound is selected from the group consisting of antibiotics, analgesics, anti-inflammatories, strong oxidizing agents, matrix metalloproteinase inhibitors, proteins, peptides, fragrances, and antifungals.

82. The composition according to claim 76 wherein said anionic biologically or chemically active compounds associated with said quaternary amine polymer is selected from the group consisting of antibiotics, analgesics, anti-inflammatories, strong oxidizing agents, matrix metalloproteinase inhibitors, proteins, peptides, fragrances, and antifungals.
